(19) United States
(12) Patent Application Publication
(10) Pub. No.: US 2022/0354527 A1
(43) Pub. Date: Nov. 10, 2022

(54) SOFT TISSUE CUTTING DEVICE WITH BOWING MECHANISM

(71) Applicant: Sonex Health, Inc., Eagan, MN (US)

(72) Inventor: DARRYL E. BARNES, EAGAN, MN (US)

(73) Assignee: Sonex Health, Inc., EAGAN, MN (US)

(21) Appl. No.: 17/741,006

(22) Filed: May 10, 2022

Related U.S. Application Data
(60) Provisional application No. 63/186,538, filed on May 10, 2021.
(60) Provisional application No. 63/186,524, filed on May 10, 2021.

(51) Int. Cl. A61B 17/32; A61B 17/14

(52) U.S. Cl. CPC A61B 17/320036; A61B 17/149

(57) ABSTRACT

A device for cutting soft tissues in the body, such as a transverse carpal ligament in a hand, includes a handle, a shaft, a cutting device on or in the shaft (e.g., a blade or radiofrequency electrodes), and a single expansion band or bowing flanges that bow out laterally to form a safe zone. The expansion band or bowing flanges is/are movable between an expanded configuration and a retracted configuration using a slidable actuator on the handle.

### CROSS REFERENCE TO RELATED APPLICATIONS

This application claims the benefit of U.S. Provisional Patent Application No. 63/186,538, filed May 10, 2021 and U.S. Provisional Patent Application No. 63/186,524, filed May 10, 2021, the disclosures of which are incorporated herein by reference in their entirety for any and all purposes.

### FIELD OF THE DISCLOSURE

The present disclosure generally relates to medical devices and methods. More specifically, the disclosure is related to a surgical device and method for cutting soft tissue, such as in a carpal tunnel release procedure

### BACKGROUND OF THE DISCLOSURE

Carpal tunnel syndrome is a condition that causes numbness, tingling and/or weakness in the hand. The condition occurs when pressure is placed on the median nerve over long periods of time, typically by the transverse carpal ligament. The median nerve runs the length of the arm, passes through a passage in the wrist called the carpal tunnel, and ends in the palm of the hand. Carpal tunnel syndrome is extremely prevalent, affecting approximately 3.7% of the general population and up to 7% of manual labor workers. The most common surgical procedure used for treating carpal tunnel syndrome, known as a carpal tunnel release procedure (“CTR procedure”), involves cutting the transverse carpal ligament to reduce pressure on the median nerve and thus alleviate symptoms. CTR procedures can be performed by accessing the carpal tunnel primarily from the outside or primarily from the inside. In the former type of procedure, an incision is made on the palm of the hand, so that the physician (e.g., a surgeon or other suitable physician) can directly visualize and cut through the transverse carpal ligament. In the latter type of procedure, the transverse carpal ligament is visualized from within the carpal tunnel using an endoscope advanced through a small incision on the palm or the wrist. This type of procedure is referred to as endoscopic carpal tunnel release (ECTR).

One concern with CTR procedures is that it can be challenging to visualize the transverse carpal ligament and nearby structures that physician does not want to damage, such as the median nerve and the ulnar artery. It can also be difficult to visualize individual anatomical variations in the carpal tunnel region. As a result, some CTR procedures can produce unsatisfactory results, due to incomplete release of the transverse carpal ligament, while others can cause unwanted injury to surrounding structures. To try to achieve better visualization, physician may sometimes create a larger incision on the palm of the hand, which can be a difficult area to heal and thus may cause a long recovery period for the patient, with lingering pain, discomfort and inability to return to work.

In recent years, physician other physicians who perform CTR procedures have used ultrasound guidance during the procedure, to better visualize the carpal tunnel region. Even with ultrasound visualization, however, there are still potential pitfalls with CTR. One concern is that the procedure involves passing a sharp cutting instrument into the carpal tunnel without direct visualization. Furthermore, the “safe zone,” in which the transverse carpal tunnel ligament can be cut without risk to the median nerve or ulnar artery, is typically very small, for example less than 3 millimeters in some patients. Placing a sharp cutting instrument in this narrow safe zone carries inherent risk of unwanted injury to tissues such as the median nerve and ulnar artery.

Thus, it would be beneficial to have improved devices and methods for treating carpal tunnel system. Ideally, such devices and methods would help improve outcomes and reduce side effects.

### SUMMARY OF THE DISCLOSURE

In one aspect of the present disclosure, a soft tissue cutting device may include a shaft, a shaft opening, a cutting blade, and a single expansion band. The shaft has multiple surfaces, and the shaft opening extends for a distance along at least one of the multiple surfaces. The cutting blade extends through and withdraws from the shaft opening. The cutting blade is located near a distal end of the soft tissue cutting device. The single expansion band generally surrounds the distal end of the soft tissue cutting device and is movable between an expanded configuration and a retracted configuration. The expansion band is configured to expand laterally outwardly relative to the cutting blade when the single expansion band is in the expanded configuration.

In another aspect of the present disclosure, a soft tissue cutting device may include a handle, a bowing actuator, a cutting blade, and an expansion band. The handle is located at a proximal end of the device, and the cutting blade is positioned near a distal end of the device. The expansion band generally surrounds the distal end of the device. The expansion band is movable between an expanded configuration and a retracted configuration. The expansion band is configured to bow outwardly relative to lateral sides of the cutting blade when the expansion band is in the expanded configuration. The bowing actuator is slidable relative to the handle. Sliding the bowing actuator such that it contacts the expansion band and exerts a force on the expansion band causes the expansion band to move into the expanded configuration.

In another aspect, a soft tissue cutting device may include a shaft, a shaft opening, a cutting blade, an elongated support, and a pair of bowing flanges. The shaft has multiple surfaces, and the shaft opening extends for a distance along at least one of the multiple surfaces. The cutting blade extends through and withdraws from the shaft opening. The elongated support has a first end, a second end, and a main portion that extends between the first and second ends. The first end has a pair of tabs. Each tab projects laterally away from the main portion of the elongated support. Each bowing flange is movable between an expanded configuration and a retracted configuration. The bowing flanges are configured to bow outwardly relative to lateral sides of the cutting blade when the bowing flanges are in the expanded configuration. A distal end of each bowing flange is attached to a respective tab of the elongated support.

In another aspect, a soft tissue cutting method may involve advancing a soft tissue cutting device to a body region, expanding an expansion band of the device radially outward, and pivoting a cutting blade of the device upward to expose a cutting surface of the cutting blade to cut soft tissue. The soft tissue cutting device includes a cutting blade and a single expansion band that bows outwardly relative to lateral sides of the cutting blade.

In another aspect, the present disclosure provides a method of cutting a transverse carpal ligament. The method involves advancing a device to a carpal tunnel region while the device is in an inactive position and cutting a transverse carpal ligament while the device is in an active position. In the inactive position, the cutting device includes a blade that is housed within a shaft of the cutting device and an expansion band that is in a retracted configuration. In the active position, the cutting blade extends through an opening in the shaft of the cutting device and the expansion band is in an expanded configuration.

In another aspect, an electrosurgical soft tissue cutting device is disclosed. The device may include a handle, and a shaft extending from the handle. The shaft may include a proximal portion, a distal portion, an upper surface, a lower surface, and a first shaft opening that extends longitudinally along the upper surface of at least part of the distal portion of the shaft. The device may further include a pair of electrodes configured for cutting soft tissue and a first expansion band proximate the distal portion of the shaft. The expansion band may be movable between an expanded configuration, in which the first expansion band bows outwardly in a lateral direction, and a retracted configuration.

In another aspect, an electrosurgical soft tissue cutting device is disclosed. The device may include a handle located at a proximal end of the electrosurgical soft tissue cutting device, a shaft extending from the handle, a pair of electrodes having a working end positioned near a distal end of the electrosurgical soft tissue cutting device, a first pair of bowing flanges, and a second pair of bowing flanges disposed on opposite sides of the pair of electrodes. The first and the second pair of bowing flanges may be movable between an expanded configuration and a retracted configuration. In the expanded configuration, the first and the second pair of bowing flanges are configured to bow outwardly relative to lateral sides of the pair of electrodes. The device may also include a bowing actuator that is slidable relative to the handle, so that sliding the bowing actuator contacts and exerts a force on the first pair of bowing flanges and the second pair of bowing flanges to cause them to move into the expanded configuration.

In another aspect, a method of cutting a transverse carpal ligament in a patient is disclosed. The method may include advancing a shaft of an electrosurgical device into a hand of the patient to position a distal end of the device at or near the transverse carpal ligament. The electrosurgical device may include a pair of electrodes at or near the distal end of the shaft, and a pair of lateral expansion bands on the shaft. The method may also include expanding the pair of lateral expansion bands such that each expansion band of the pair of expansion bands bows radially outwardly relative to lateral sides of the pair of electrodes in opposite directions, positioning the shaft such that the transverse carpal ligament lies within or adjacent to the pair of electrodes, and electrically charging the pair of electrodes to transect the transverse carpal ligament.

These and other aspects and embodiment are described in further detail below, in relation to the attached drawing figures.

### BRIEF DESCRIPTION OF THE DRAWINGS

FIG. 1 is a perspective view of a soft tissue cutting device, according to one embodiment.

FIG. 2 is a side view of the soft tissue cutting device of FIG. 1.

FIG. 3 is a top view of the soft tissue cutting device of FIGS. 1 and 2, showing an expansion band moving between a retracted configuration and an expanded configuration.

FIG. 4 shows an expansion band, according to one embodiment.

FIG. 5 shows an expansion band, according to one embodiment.

FIG. 6 is a side view of a cutting blade and a push rod, according to one embodiment.

FIG. 7 is a side view of a cutting blade and a push rod, according to another embodiment.

FIG. 8 is a side view of an extension tube, according to one embodiment.

FIG. 9 is a front view of a cutting blade of one embodiment.

FIG. 10 is a top view of a handle of one embodiment.

FIG. 11 is a perspective view of an electrosurgical soft tissue cutting device, according to one embodiment.

FIG. 12 is a side view of the electrosurgical soft tissue cutting device of FIG. 11.

FIG. 13A is a top view of the electrosurgical soft tissue cutting device of FIGS. 11 and 12, showing the expansion band moving between a retracted configuration and an expanded configuration.

FIG. 13B is a side view of the electrosurgical soft tissue cutting device of FIGS. 11-13A, showing a lower expansion band moving between a retracted configuration and an expanded configuration.

FIG. 14 is a side view of a pair of electrodes and a push rod, according to one embodiment.

FIG. 15 is a side view of both a pair of electrodes and a push rod, according to another embodiment.

FIG. 16 is a front view of a pair of electrodes of one embodiment.

FIG. 17 is a schematic view of an electrosurgery system.

FIG. 18A is a side view, showing an exemplary bowing flange in an active expanded position and an exemplary bowing flange in a non-active and non-expanding position, according to one embodiment.

FIG. 18B is an enlarged illustration of region A-A of FIG. 18A.

FIGS. 18C and 18D illustrate an alternative embodiment for moving the bowing flanges to an expanded configuration.

FIG. 19 is a perspective view of a bowing flange, according to one embodiment.

FIG. 20A is a top view of the bowing flange of FIG. 19.

FIG. 20B is a side view of the bowing flange of FIGS. 19 and 20A.

FIG. 20C is a front view of the bowing flange of FIGS. 19-20B.

FIG. 21A is a side view of an exemplary bowing flange in a non-expanded position.

FIG. 21B is an enlarged illustration of region B-B of FIG. 21A.

FIG. 22A is a perspective view of an exemplary bowing flange and bowing actuator, according to one embodiment.

FIG. 22B is a perspective view of a blade actuator and a bowing actuator in a first position, according to one embodiment.

FIG. 22C is a perspective view of a blade actuator and a bowing actuator in a second position, according to one embodiment.

FIG. 22D is a perspective view of a blade actuator and a bowing actuator in a third position, according to one embodiment.

FIG. 23 is a perspective view of a bowing system, according to one embodiment.

### BRIEF DESCRIPTION OF DISCLOSED EMBODIMENTS

The devices 10, 700 of the present disclosure may be understood more readily by reference to the following detailed description of the embodiments, taken in connection with the accompanying drawing figures, which form a part of this disclosure. This application is not limited to the specific devices, methods, conditions, or parameters described and/or shown herein, and the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” the particular value forms another embodiment. All spatial references, such as, for example, proximal, distal, horizontal, vertical, top, upper, lower, bottom, left and right, are for illustrative purposes only and can be varied within the scope of the disclosure. For example, the references “upper” and “lower” are relative and used only in the context to the other and are not necessarily “superior” and “inferior”.

As used herein, “electrosurgery” may refer to the application of a high-frequency, alternating polarity, electrical current to a biological tissue to cut, coagulate, desiccate, or fulgurate tissue, unless the context clearly indicates otherwise. Various embodiments may principally involve an electrosurgery device including at least one electrode configured to use radio frequency (RF) signals for performing a transection to patient tissue. While the application describes the use of radio frequency signals for electrosurgery, other types of energy signals, such as, without limitation, microwave signals, ultrasound signals, laser energy signals, or the like, may be similarly used, without deviating from the principles of this disclosure. Radio frequency signals may be generated using an RF electrosurgical unit (ESU), for example. Electrosurgical units and their operation are well known in the art and will not be discussed at length in this application. Several known example electrosurgical units include: Medtronic's ValleyLab Force series, Johnson & Johnson's Ethicon series, Entermed's Enterwave series, Covidien's ForceTriad Energy Platform, Bovie Bantam series, and Erbe's Erbotom series.

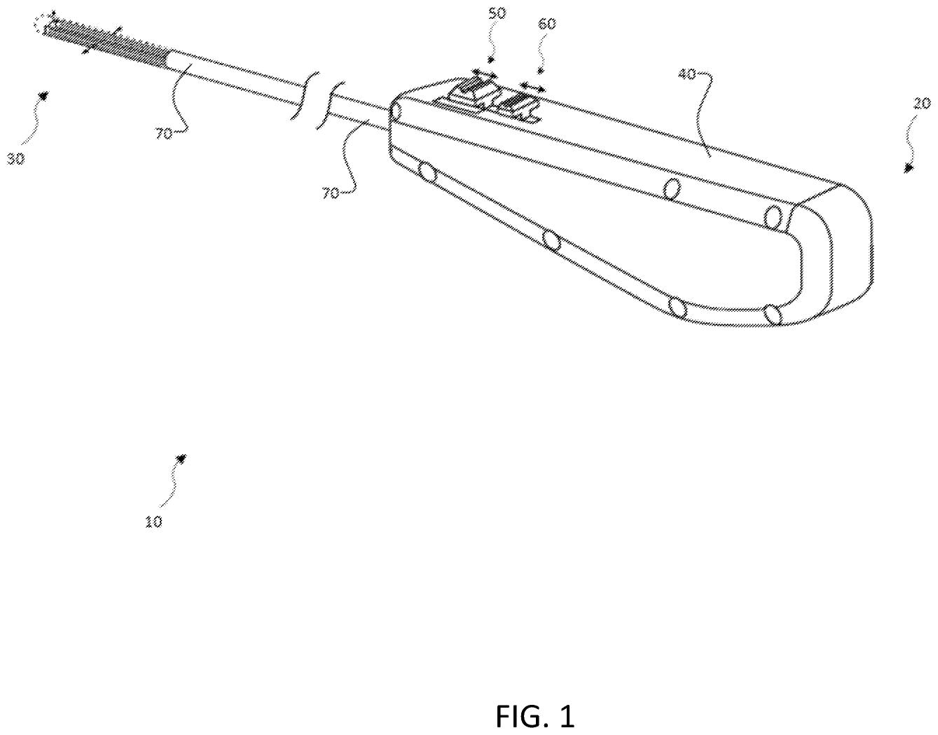

Referring generally to FIGS. 1-16, various embodiments of the disclosure provide a soft tissue cutting device 10. The soft tissue cutting device 10 can be used to cut any desired soft tissue in a body. In some embodiments, the soft tissue cutting device 10 is a transverse carpal ligament cutting device, intended to cut a transverse carpal ligament in a carpal tunnel region.

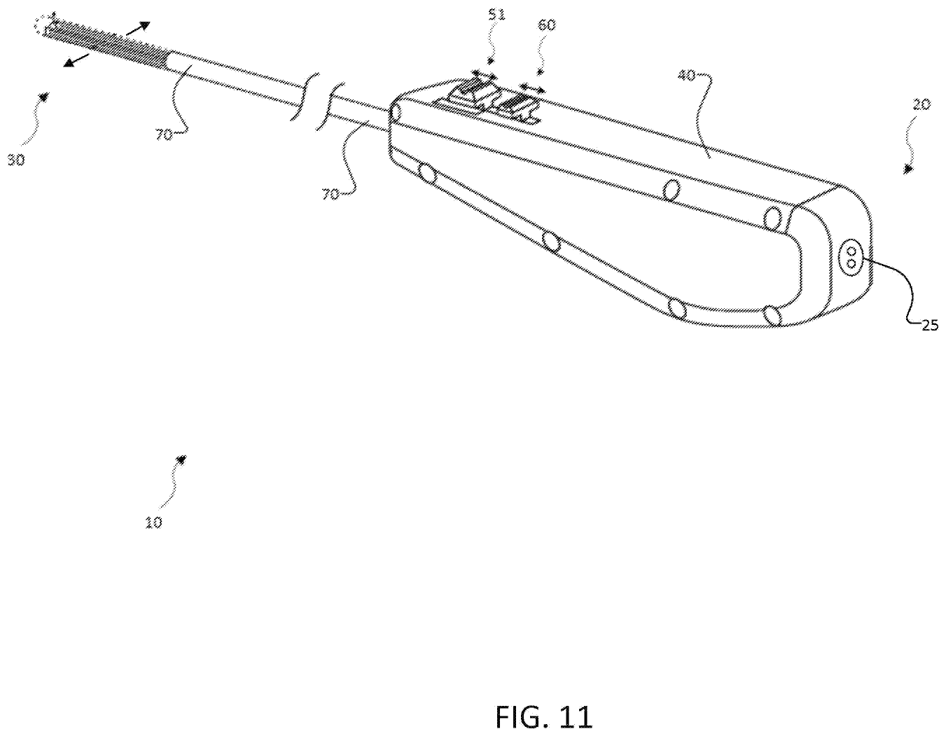

The soft tissue cutting device 10 may be a mechanical and/or electrical surgical device. FIGS. 1-10 depict a mechanical soft tissue cutting device 10 configured to expose a blade 130 to soft tissue. FIGS. 11-16 depict an electrosurgical soft tissue cutting device 10 configured to expose one or more electrodes 131 to soft tissue. A cutting element may refer to a blade 130, one or more electrodes 131, and/or other suitable cutting features configured to cut soft tissues of a subject (e.g., a patient or other suitable subject).

The soft tissue cutting device 10 includes a proximal end 20 and a distal end 30. The distal end 30 is the working end that inserts into the body region of a patient. As will be explained in greater detail below, the proximal end 20 includes one or more actuators 50, 60 that control various functions on the distal end 30, such as cutting soft tissue in a body and/or expanding a “safe zone.”

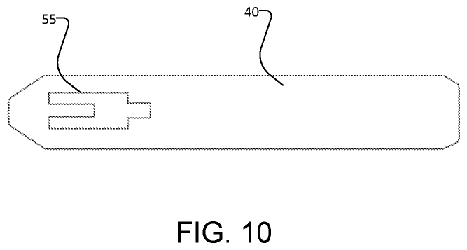

In some embodiments, the proximal end 20 includes a handle 40. In embodiments of this nature, the handle 40 can include one or more openings 55 (see FIG. 10), configured to receive the one or more actuators 50, 60 therein. This arrangement allows the physician to grip the handle 40 while operating the one or more actuators 50, 60 using the physician's thumbs and/or fingers.

A shaft 70 is coupled to the handle 40 and extends toward the distal end 30 of the tissue cutting device 10. The handle 40 can be coupled to the shaft 70 using a variety of different configurations. In some embodiments, the handle 40 and the shaft 70 have a permanent junction. For example, the handle 40 and the shaft 70 can have a set angular junction or a set straight junction. In other embodiments, the handle 40 and the shaft 70 have an adjustable junction that can be adjusted to accommodate physician preference. In other embodiments, the handle 40 and the shaft 70 have a rotatable junction that can be rotated to accommodate physician preference. For example, the handle 40 and the shaft 70 can have a junction that is adjustable in length or angulation or rotation. In other embodiments, the handle 40 and the shaft 70 can have a removable junction, so the handle 40 and the shaft 70 are removable from one another.

The shaft 70 can have any desired cross-sectional shape. In some embodiments, the shaft 70 has a circular cross-sectional shape. In other embodiments, the shaft 70 has a non-circular cross-sectional shape. For example, in some embodiments, the shaft 70 has a squared cross-sectional shape, rectangular cross-sectional shape, or squared with round edges cross-sectional shape.

As the shaft 70 extends from the proximal end 20 toward the distal end 30, the shaft 70 can maintain the same cross-sectional shape, or it can assume a different cross-sectional shape. For example, in some embodiments, the shaft 70 maintains a circular cross-section as it extends from the proximal end 20 to the distal end 30. In other embodiments, the shaft 70 changes from a circular cross-section to a non-circular cross section as it extends from the proximal end 20 to the distal end 30. In yet other embodiments, the shaft 70 maintains a non-circular cross section as it extends from the proximal end 20 to the distal end 30. In still other embodiments, the shaft 70 changes from a non-circular cross-section to a circular cross-section as it extends from the proximal end 20 to the distal end 30.

The shaft 70 can also be provided as a single piece or as multiple pieces. In some embodiments, the shaft 70 extends from the proximal end 20 to the distal end 30 as a single piece. In other embodiments, the shaft 70 extends from the proximal end 20 to the distal end 30 as multiple pieces. The shaft 70 is also formed of any desired medically acceptable material. The shaft 70 can have any desired size that is suitable for the medical procedure being performed. As the shaft 70 extends from the proximal end 20 to the distal end 30, it can maintain the same diameter or it can assume a different diameter. In some embodiments, the shaft 70 increases in diameter as it extends from the proximal end 20 to the distal end 30. In other embodiments, the shaft 70 decreases in diameter as it extends from the proximal end 20 to the distal end 30.

At the distal end 30 of the soft tissue cutting device 10, the shaft 70 has a size and cross-sectional shape that is suitable for being inserted into the body region of interest. The shaft 70 extends longitudinally along a longitudinal axis. The shaft 70 also has a top surface 80, side surfaces 90, and a bottom surface 100. Each of the top surface 80, side surfaces 90, and bottom surface 100 can be made up of a single wall or multiple of walls. Additionally, each wall of the shaft 70 can be a straight wall or a curved wall. In some embodiments, the shaft 70 is circular, such that the top quarter of the circle forms the top surface 80, the bottom quarter of the circle forms the bottom surface 100, and the remaining quarters form the side surfaces 90. In other embodiments, the shaft 70 is non-circular. The shaft 70 also has a surface that is designed to be positioned adjacent (or even in direct contact with) the soft tissue being cut, for example. In some embodiments, the top surface 80 is the surface designed to be positioned adjacent the soft tissue.

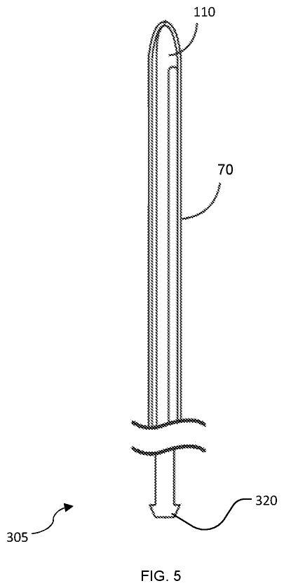

The distal end 30 of the tissue cutting device 10 includes a tip 110 (see FIG. 5). The tip 110 is the distal-most end of the tissue cutting device 10. In some embodiments, the tip 110 is an extension of the shaft 70. In other embodiments, the tip 110 is a separate piece that is positioned on a distal end of the shaft 70. The tip 110 can have any size or shape that guides the distal end 30 of the tissue cutting device 10 to the body region. In some embodiments, the tip 110 has a rounded configuration, an ovoid configuration, a pointed configuration, or a conical configuration. In such configurations, the geometry of the tip may help guide the device 10 during surgery. In some embodiments, the tip 110 is an echogenic tip that is sized and shaped to house an ultrasound probe, camera or one or more optical fiber elements to transmit light to or from a distal region of the tip 110 to the proximal end 20 of the tissue cutting device 10.

The shaft 70 also includes at least one shaft opening 120. Shaft opening 120 may be designed to facilitate the blade extending or pivoting therethrough (as shown by representation via arrows). The shaft opening 120 extends along a longitudinal axis for a distance longitudinally along the shaft 70. The shaft opening 120 also extends along a surface of the shaft 70 that is designed to be positioned adjacent to or in direct contact with the soft tissue being cut. In some embodiments, the shaft opening 120 extends longitudinally along the top surface 80 and two side surfaces 90, so that the shaft opening 120 is open superiorly, medially, and laterally. This results in an “open” shaft opening. In other embodiments, the shaft opening 120 extends longitudinally only along the top surface 80, so that the shaft opening 120 is only open superiorly. This results in a “closed” or “semi-closed” shaft opening.

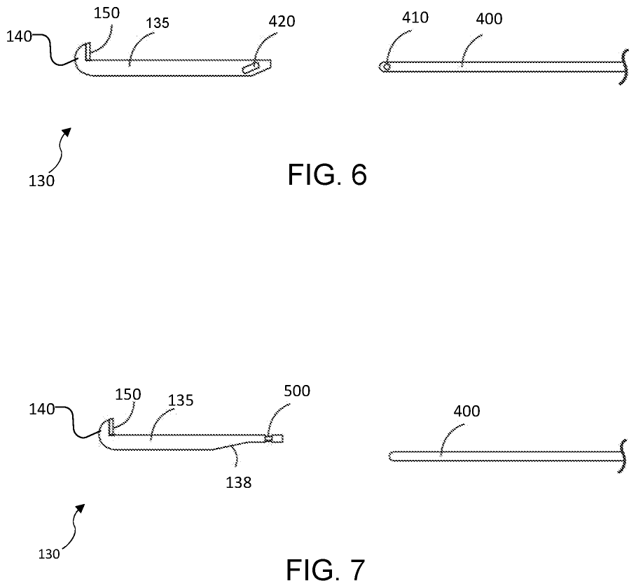

Referring to FIGS. 6 and 7, the shaft 70 also houses a blade 130. The blade 130 includes a blade shaft 135 and a blade working end 140. The blade shaft 135 has a cutting surface 150 configured to cut the soft tissue. The cutting surface 150 can have any desired configuration that allows the cutting surface 150 to cut soft tissue. In some embodiments, the cutting surface 150 has a sharp straight edge. In other embodiments, the cutting surface 150 has a sharp curved edge. In yet other embodiments, the cutting surface 150 has an angulated, toothed, serrated, or sawed edge.

The cutting surface 150 can be positioned on any surface of the cutting blade 130. In some embodiments, the blade working end 140 is linear and the cutting surface 150 is provided on a distal edge of the blade working end 140. In certain other embodiments, the blade working end 140 is linear and the cutting surface 150 is provided on a top edge of the blade working end 140. In other embodiments, the blade working end 140 is configured as a hook and the cutting surface 150 is provided on an interior surface of the hook. In still yet other embodiments, the blade 130 has two cutting surfaces 150 that allow the blade 130 to cut when pushed or pulled through soft tissue.

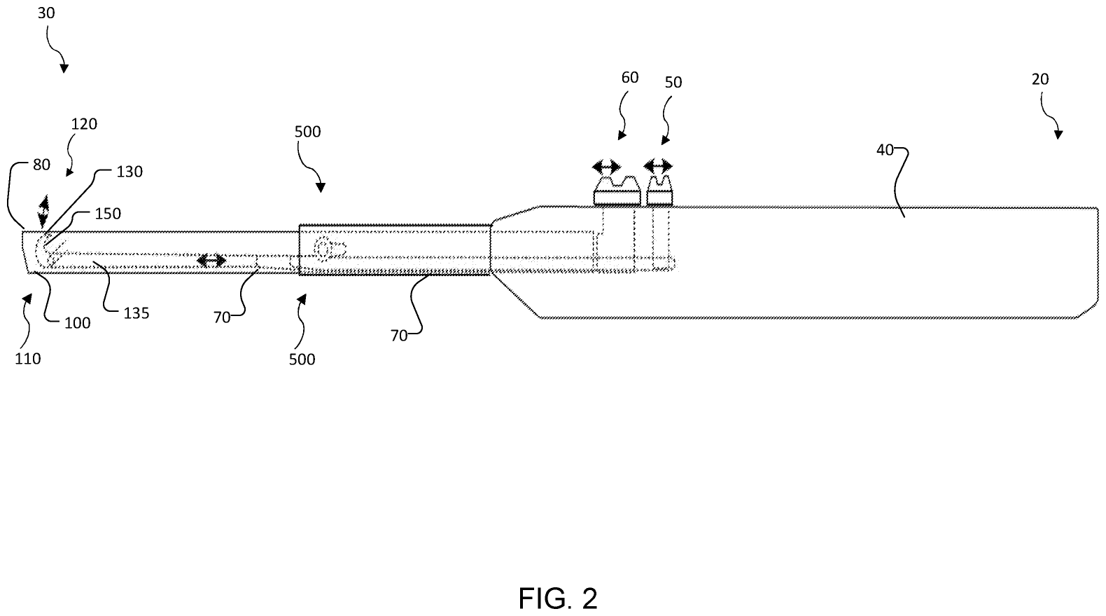

The blade 130 is movable with respect to the shaft 70. In some embodiments, the cutting edge 150 is movable to an active (exposed) position where blade 130 may extend through an opening in the shaft (represented by arrows in FIG. 2), for example through opening 120. Similarly, the cutting edge 150 is movable to a non-active (non-exposed) position where blade 130 may be hidden and/or protected within shaft 70. This dual positioning of the blade 130 may allow the cutting blade 130 to be safely inserted into the body when the cutting blade 130 is in the inactive position. In particular, when the soft tissue cutting device 10 is in the active position, the cutting edge 150 of the blade 130 extends through the shaft opening 120 and is exposed for cutting tissue. In contrast, when the soft tissue cutting device 10 is in the inactive position, i.e., blade 130 does not extend through opening 120, the blade 130 is housed within the shaft 70 so as to prevent the cutting edge 150 of the blade 130 from coming into contact with soft tissue. The blade 130 can be moved from the inactive position to the active position when it is desired to cut soft tissue, e.g., by an actuator or the like. Then, after the cutting has been performed, the blade 130 can be moved from the active position to the inactive position so as to house the blade 130 within the shaft 70, for example by an actuator 50.

Actuator 50 may be a blade actuator configured to move the cutting blade 130 between the active position and the inactive position as explained above. In some embodiments, the actuator 50 is a slidable actuator configured to slide along a portion of the handle 40. For example, a physician may slide actuator 50 forward and/or backward using one or more fingers or thumbs. In some embodiments, actuator 50 includes a lock configured to lock the blade 130 in a desired position. A lock is not required in all embodiments.

In some embodiments, the blade 130 may be pivotable with respect to the shaft 70. In some instances, the blade 130 is pivotable between the active position and the inactive position. As described in greater detail below, pivoting of the blade 130 can be accomplished in various ways.

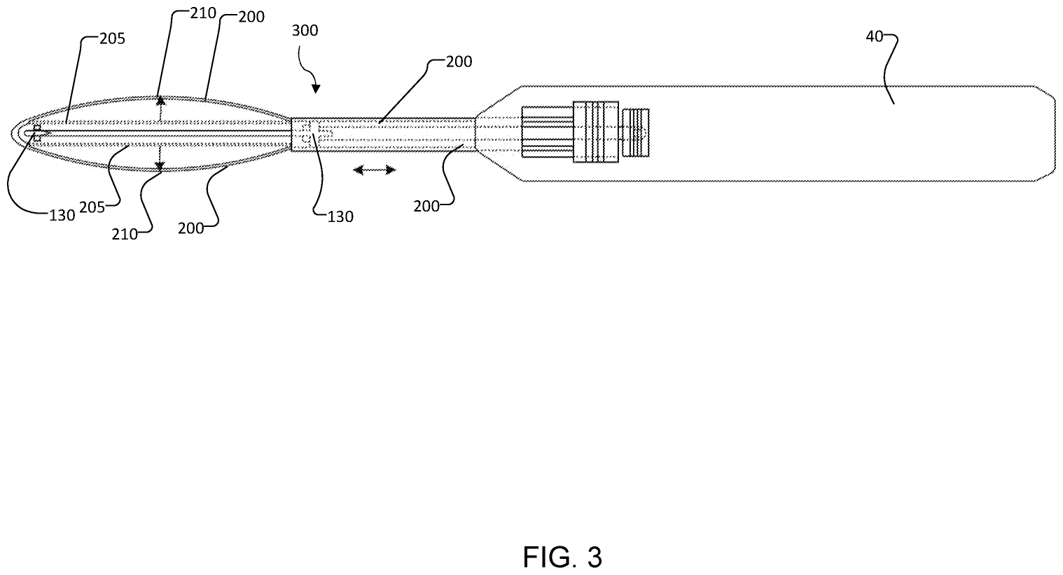

The device 10 may also include at least one expansion band 200 that generally surrounds the distal end 30 of the device 10 (see FIG. 3). FIG. 3 illustrates a top down view where two expansion bands 200 that are movable between a retracted configuration 205 and an expanded configuration 210. As illustrated, the expanded configuration 210 of expansion band 200 is shown in solid lines and the retracted configuration 205 is shown in dashed lines for ease of understanding. While FIG. 3 illustrates two expansion bands, the disclosure is not so limiting, and any number of expansion bands (e.g., one or three) may be used. As shown in FIG. 3, a portion of the expansion band 200 expands radially and outwardly relative to lateral sides of the cutting blade 130 when the expansion band 200 is in the expanded configuration 210. In more detail, the expansion band 200 bows outwardly when in the expanded configuration 210 so as to expand laterally away from the cutting blade 130 and push tissue away from lateral sides of the cutting blade 130. This expansion is illustratively shown by arrows in FIG. 3. In some embodiments, the expansion band 200 expands only laterally (and not upwardly or downwardly) relative to the cutting blade 130. In some other embodiments, two lateral expansion bands may expand laterally and a lower expansion band may expand downwardly relative to the cutting blade 130, for example. In other embodiments, the expansion band 200 expands laterally, upwardly, and downwardly relative to the cutting blade 130 like a balloon undergoing inflation, for example. In disclosed embodiments, each expansion band 200 may comprise plural elongated bands. In some embodiments, each expansion band 200 may comprise only a single elongated band.

The expansion band 200 may help to anchor the soft tissue cutting device 10 within the body region to provide stability during cutting of the soft tissue. In addition, when the expansion band 200 is in the expanded configuration 210, it may help to push nearby at-risk structures away from the soft tissue cutting device 10 during cutting. In this way, expansion band 200 may expand the “safe zone” in advance of and while the cutting blade 130 performs cutting so as to ensure that only the desired soft tissue is cut and that nearby at-risk structures are not cut.

As discussed above, the tissue cutting device 10 may also include at least one actuator. For example, actuator 60, also referred to herein as a bowing actuator, is coupled to the expansion band 200 such that the movement of the actuator 60 is configured to move the expansion band 200 between the retracted configuration 205 and the expanded configuration 210 (as shown in FIG. 3). In some embodiments, the actuator 60 is a slidable actuator configured to slide along a portion of the handle 40. A physician can slide the actuator 60 using one or more fingers or thumbs, similar to actuator 50. The actuator 60 is configured to slide toward the expansion band 200 such that the actuator 60 contacts the expansion band 200 and applies a force to the expansion band 200. When sufficient force is applied to the expansion band 200, the expansion band 200 bows outwardly so as to move the expansion band 200 from the retracted configuration 205 to the expanded configuration 210. In some embodiments, the actuator 60 includes a lock to lock the expansion band 200 in a desired position. In some embodiments, each expansion band may have its own actuator (not illustrated), and/or one or more expansion bands may be collectively controlled using a single actuator.

In some embodiments, a physician can deploy the cutting blade 130 only after the expansion band 200 has been moved to the expanded configuration 210. This is a safety feature designed to prevent the cutting blade 130 from being deployed (i.e., exposed) if the expansion band 200 has not been expanded.

In disclosed embodiments, the expansion band 200 is not inflatable, and thus does not expand via an inflatable mechanism. Rather, in such embodiments, the expansion band 200 is made from a material that is deformable so as to move the expansion band 200 into the expanded configuration 210 when a sufficient contact force is applied thereto. When the force ceases to be applied to the expansion band 200, the expansion band 200 returns to the retracted configuration 205 naturally as the expansion band 200 may itself provide a biasing force returning to the neutral position (non-expanded position). In some embodiments, the expansion band 200 is made from a flexible and moldable metal or plastic. However, it is envisioned that alternative materials for the expansion band 200 can also be used, e.g., an elastomeric material.

In some embodiments, the tissue cutting device 10 may include an extension tube 300. The extension tube 300 may be attached to the handle 40 and surround a portion of the expansion band 200. For example, as shown in FIG. 3 a portion of expansion band 200 is shown within an inside cavity of extension tube 300 in skeleton lines. A portion of the expansion band 200 located near the distal end 30 of the tissue cutting device 10 extends beyond, and thus is not positioned within, the extension tube 300. Thus, in some embodiments, when the actuator 60 is slid toward the distal end 30, a compressive force is applied to the expansion band 200 resulting in, only the portion of the expansion band 200 that extends beyond the tube 300 being able to expand to the expanded configuration 210 (because the remaining portion of the expansion band 200 is confined within a cavity the extension tube 300). This arrangement may be advantageous for some surgical procedures and transections in relatively tight locations. In some embodiments, the size of the sheath may also be adjustable to accommodate various surgical techniques with only one device 10. In some embodiments, the device 10 can be devoid of any structure surrounding the expanded portion of the expansion band 200 when the expansion band 200 is in the expanded configuration 210. The extension tube 300 may comprise metal, plastic, or any other suitable material. Additionally, the extension tube 300 may be removable for cleaning and/or coupling and uncoupling of blade 130.

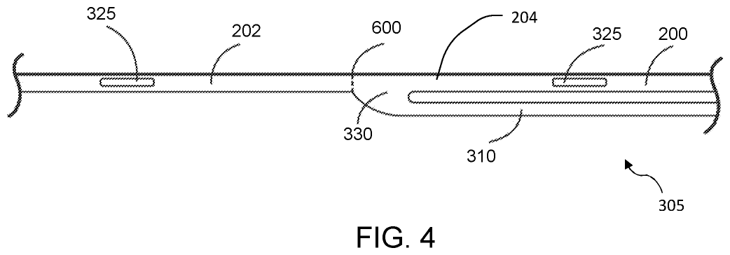

As shown in FIG. 5, in some embodiments, the expansion band 200 can include an anchor 305 configured to attach the expansion band 200 to the extension tube 300. As shown in FIGS. 4 and 5, the anchor 305 can comprise an anchor arm 310 and an anchor portion 320. The anchor portion 320 may directly attach the expansion band 200 to the extension tube 300. In some embodiments, as shown in FIG. 4, a junction 330 is formed between the anchor arm 310 and portion 204 of the expansion band 200.

Referring to FIGS. 6 and 7, the tissue cutting device 10 may also include a push rod 400. The push rod 400 may be positioned in the shaft 70 adjacent the actuator 50, for example. The push rod 400 may comprise metal, plastic, or any other suitable material. In some embodiments, the push rod 400 includes a pin 410, and the blade shaft 135 includes a slot 420 located in the blade shaft 135. This arrangement enables the cutting blade 130 and blade shaft 135 to pivot upwardly when the actuator 50 is moved distally, which in turn causes the cutting blade 130 to move into the active position as explained hereinabove. This non-limiting embodiment is shown in FIG. 6.

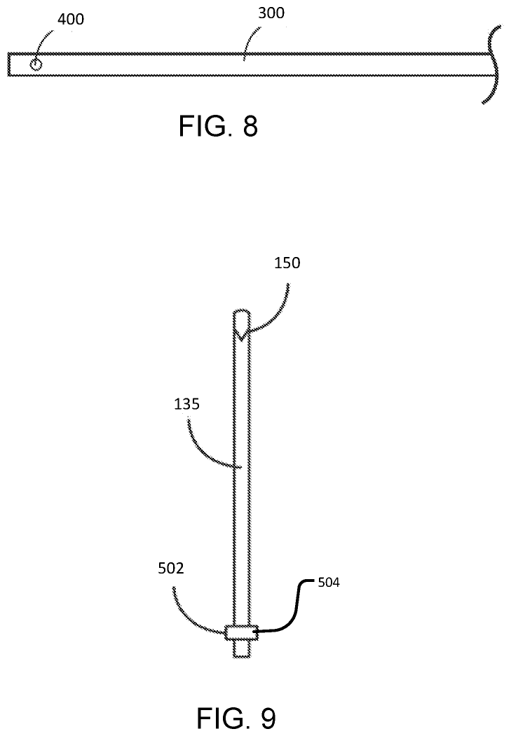

In certain other embodiments, as shown in FIG. 7, the cutting blade 130 is pivotally coupled to both the expansion band 200 and the extension tube 300 via a pivot point 500. In some embodiments, the pivot point 500 and the cutting blade 130 are integrally formed. In other embodiments, the pivot point 500 and the cutting blade 130 can be distinct structures that are coupled together by a hinge, pin, ball and socket, or the like. In some embodiments, the pivot point 500 is a projection comprising a thin, flattened portion of the blade shaft 135 (see FIG. 9). The pivot point 500 can be housed within the extension tube 300 and pivot within a slot 325 located in the expansion band 200 (see FIGS. 4 and 5) and also within a hole 510 located in the extension tube 300 (see FIG. 8). Such a structural arrangement allows the cutting blade 130 to pivot between the active position and the inactive position as previously explained.

The expansion band 200 can also include a fold point 600 located between first and second portions 202, 204 of the expansion band 200 (see FIG. 4). During manufacture of the tissue cutting device 10, the expansion band 200 can be folded along the fold point 600 in such a manner so as to align the two slots 325. In embodiments of this nature, opposing ends 502, 504 of the pivot point/projection 500 extend through a respective one of the aligned slots 325. In such embodiments, the aligned slots 325 may also be aligned with (e.g., overlay) the hole 510, which may facilitate the expansion band 200 in moving toward the distal end 30 of the tissue cutting device 10. In some embodiments, the projection 500 may not pivot when the expansion band 200 moves toward the distal end 30 of the tissue cutting device 10.

As shown in FIG. 7, the blade shaft 135 may include a sloped region 138 that slopes upwardly in a direction toward the projection 500. When the actuator 50 is moved in the distal direction relative to the device 10, the push rod 400 is also pushed distally so as to contact the blade shaft 135. This in turn causes the push rod 400 to contact and apply a force along the sloped region 138 of the blade shaft 135. When sufficient force is applied to the blade shaft 135, a distal end of the cutting blade 130 moves upward to move the cutting blade 130 into the active position.

In some embodiments, the distal end 30 also includes multiple suction openings (not illustrated). In some embodiments, the suction openings can be provided along a shaft surface that is designed to be positioned adjacent to or in direct contact with the soft tissue being cut. In many embodiments, the suction openings may be provided along a top surface of the shaft 70. The suction openings can have any desired configuration. For example, in some embodiments, the suction openings are circular holes. In other embodiments, the suction openings are slots. In yet other embodiments, the suction openings are provided as a single elongated slot that extends along a longitudinal axis of the shaft 70. In other embodiments still, a combination of circular holes, slots, and elongated slots are employed.

Suction may be applied to the suction openings to suck fluid, gases and/or solids suspended in fluid through the suction openings. This may cause soft tissue near the suction openings to move closer to the surface of the shaft 70. Suction can be applied to the suction openings using any desired mechanism, for example a vacuum, air compressor, or the like. In some embodiments, the shaft 70 includes one or more conduits operably coupled to the suction openings to apply suction to the suction openings. In some embodiments, a single conduit is used to apply suction to the suction openings. In other embodiments, multiple conduits can be used. For example, a first conduit can apply suction to some of the suction openings, and a second conduit can apply suction to other of the suction openings. For example, in the previously disclosed embodiment having distinct types of suction openings a first conduit may apply a suction force to circular openings at the tip of the device and a second conduit may apply a suction force to an elongated slot opening. Any arrangement of conduits can be used, according to various embodiments, and such conduits may be independently operated.

As referred to above, FIGS. 11-16 depict features of an electrosurgical soft tissue cutting device 10. The soft tissue cutting device 10 including electrosurgical cutting features is configured similarly to the soft tissue cutting device 10 including a blade 130 discussed above, but with electrodes 131 in addition to or as an alternative to the blade 130, and discussion of similar features will not be repeated.

The electrosurgical soft tissue cutting device 10 may be any suitable type of soft tissue cutting device having electrosurgical cutting components, e.g., a bipolar radiofrequency soft tissue cutting device or a monopolar electrosurgical device. In monopolar electrosurgical devices, electrical energy of a certain polarity may be provided to one or more electrodes of device 10. A separate return electrode may be electrically coupled to a patient. In bipolar radiofrequency electrosurgical tools, one or more electrodes may be electrically coupled to a source of electrical energy of a first polarity and one or more other electrodes may be electrically coupled to a source of electrical energy of a second polarity that is opposite to first polarity. In this way, bipolar radiofrequency electrosurgical tools can deliver electrical signals to a focused tissue area with a reduced risk of patient injuries

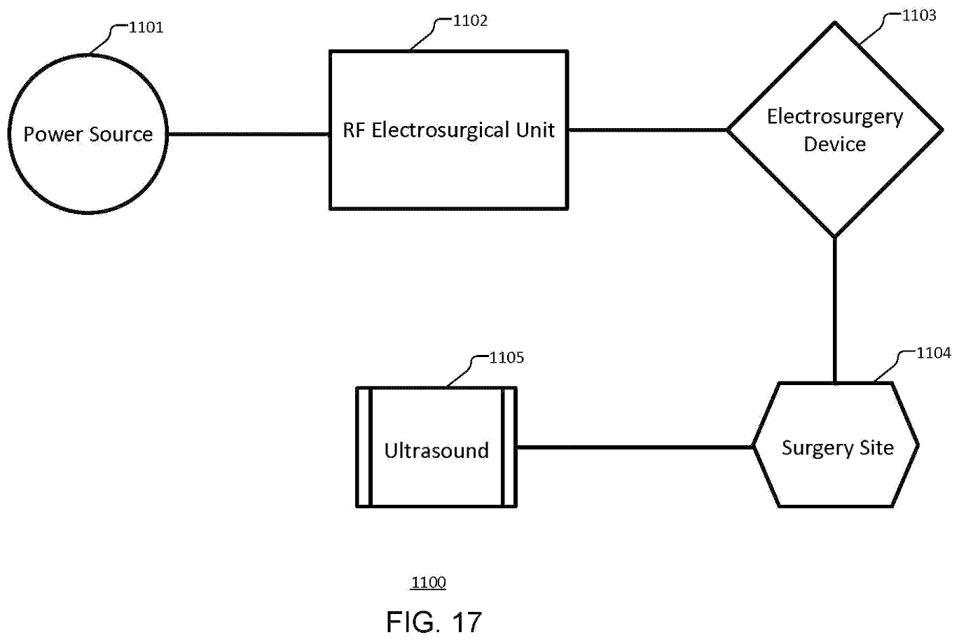

As depicted in FIG. 11, in some embodiments the proximal end 20 of the cutting device 10 may include a connection port 25. Connection port 25 may be configured to electrically connect electrosurgical soft tissue cutting device 10 with an electrosurgical unit (ESU) 1102 (see FIG. 17 schematic), for example. In various embodiments, an ESU 1102 may supply electrosurgical soft tissue cutting device 10 with a high-frequency (radio frequency) alternating polarity, electrical current.

The shaft 70 of the cutting may include at least one shaft opening 120 designed to expose electrodes 131 to facilitate tissue coming into contact with electrodes 131 for a transection, for example. The shaft opening 120 extends along a longitudinal axis for a distance longitudinally along the shaft 70. The shaft opening 120 also extends along a surface of the shaft 70 that is designed to be positioned adjacent to or in direct contact with the soft tissue being cut. In some embodiments, a first shaft opening 120 extends longitudinally along the top surface 80 and two opposing shaft openings 120 extend laterally from side surfaces 90. For example, a first shaft opening 120 exposes the electrodes 131, a second shaft opening expose first lateral side and a third shaft opening exposes a second lateral side opposite the first lateral side. In various embodiments, these shaft openings 120 may open superiorly, medially, and laterally. This results in an “open” shaft configuration, for example. In other embodiments, the shaft opening 120 extends longitudinally only along the top surface 80, so that the shaft opening 120 is only open superiorly. This results in a “closed” or “semi-closed” shaft opening.

Referring to FIGS. 6 and 7, the shaft 70 may house a pair of electrodes 131. Each electrode 131 of the pair of electrodes 131 may be charged with an opposite polarity. The pair of electrodes 131 may optionally be joined by an insulating material such as body portion 133. In some embodiments, body portion 133 may extend longitudinally along the length of electrodes 131 and structurally reinforce electrodes 131 such that are rigid and/or substantially rigid. As used herein, the term substantially rigid encompasses a meaning allowing some deflection while still remaining rigid enough to support its own weight without deflecting.

In various embodiments, body portion 133 may be configured to quickly couple and uncouple from components of the device 10 so that the electrodes 131 may be replaced. For example, some embodiments may use a single use pair of electrodes 131 configured for the particular type of surgery being performed. For example, electrodes 131 may be configured in a loop configuration, a hook configuration, and/or a straight configuration depending on the surgery type. In the example embodiment, a distal most end of the electrodes 131 may define a working end 141. Working end 141 may be understood as an open space between the tip of each electrode 131 where a circuit may be completed through a patient's tissue during operation. For example, a particular target tissue to be transected, cut, coagulated, desiccated, or fulgurated may be positioned between or proximate to the working end 141 of a rigid and/or substantially rigid pair of electrodes 131. When electrodes 131 are electrically charged the particular target tissue may complete the circuit and therefore be transected and/or vaporized, for example.

In some embodiments, the pair of electrodes 131 may be movable between an active (exposed) position and an inactive (covered) position. For example, a tip portion of the pair of electrodes 131 may be covered by a sheath to facilitate entry into a patient's body and the sheath may be removed when it is desired to transect a tissue as explained above. Similarly, working end 141 may be movable to a non-active (non-exposed) position where the tip portion of the pair of electrodes 131 may be hidden and/or protected within shaft 70. This dual positioning of the pair of electrodes 131 may allow the pair of electrodes 131 to be safely inserted into the body of a patient in the inactive position (i.e. retracted within shaft 70). In particular, when the electrosurgical soft tissue cutting device 10 is in the active position, the working end 141 of the pair of electrodes 131 extends through the shaft opening 120 and is exposed for transecting, cutting, coagulating, desiccating, or fulgurating. In contrast, when the electrosurgical soft tissue cutting device 10 is in the inactive position, i.e., pair of electrodes 131 does not extend through opening 120, the pair of electrodes 131 is housed within the shaft 70 so as to prevent the working end 141 from being capable of transecting, cutting, coagulating, desiccating, or fulgurating tissue. The pair of electrodes 131 can be moved from the inactive position to the active position when it is desired to cut soft tissue, e.g., by an actuator or the like that moves them into position. Then, after the working end 141 has been moved into position, the pair of electrodes 131 can be charged by the ESU, for example.

In various embodiments, a physician may activate the pair of electrodes 131 by an actuator 51 and charge the pair of electrodes 131 by the actuator 51. For example, a physician may slide actuator 51 forward and/or backward to electrically charge electrodes 131. Additionally, a physician may depress a button on actuator 51 to electrically charge electrodes 131, i.e., to form a circuit that loops from a first electrode 131 through the patient tissue and back through the second electrode 131. In at least one embodiment, actuator 50 must be first slid into an active position that allows a physician to depress a button to charge electrodes 131. For example, the electrodes 131 may only be charged by depressing the button after the actuator 51 is slid into an active position.

Actuator 51 may be configured to move the pair of electrodes 131 between the active position and the inactive position as explained above. In some embodiments, the actuator 51 is a slidable actuator configured to slide along a portion of the handle 40. A physician can slide the actuator 51 using one or more fingers or thumbs. In various embodiments, actuator 51 is slidable and depressible. For example, actuator 51 may be slidable forward and backward to move electrodes 131 between the active and inactive position and also include a depressible button or the like to electrically charge electrodes 131. In other embodiments, actuator 51 may only be slidable forward and backward and a physician may charge electrodes 131 by activating a foot pedal or the like as is consistent with the various example ESU's listed above. In some embodiments, and as a safety feature, actuator 51 includes a locked position and the actuator 50 must be in the locked position in order for electrodes 131 to carry an electrical current. A lock is not required in all embodiments to effectuate this safety concept.

In some embodiments, the pair of electrodes 131 may be pivotable with respect to the shaft 70. In some instances, the pair of electrodes 131 is pivotable between the active position and the inactive position. As described in greater detail below, pivoting of the pair of electrodes 131 can be accomplished in various ways.

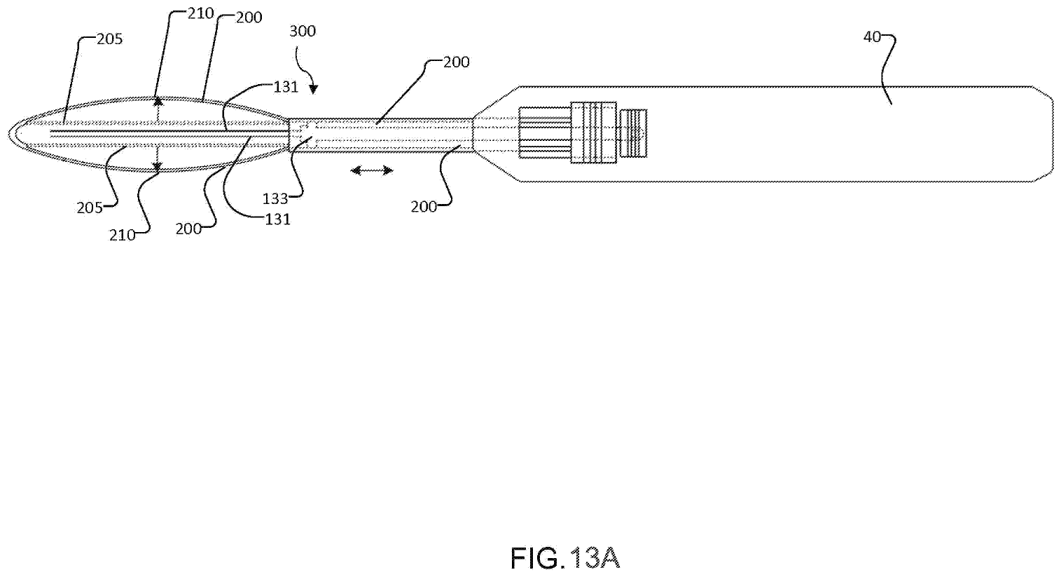

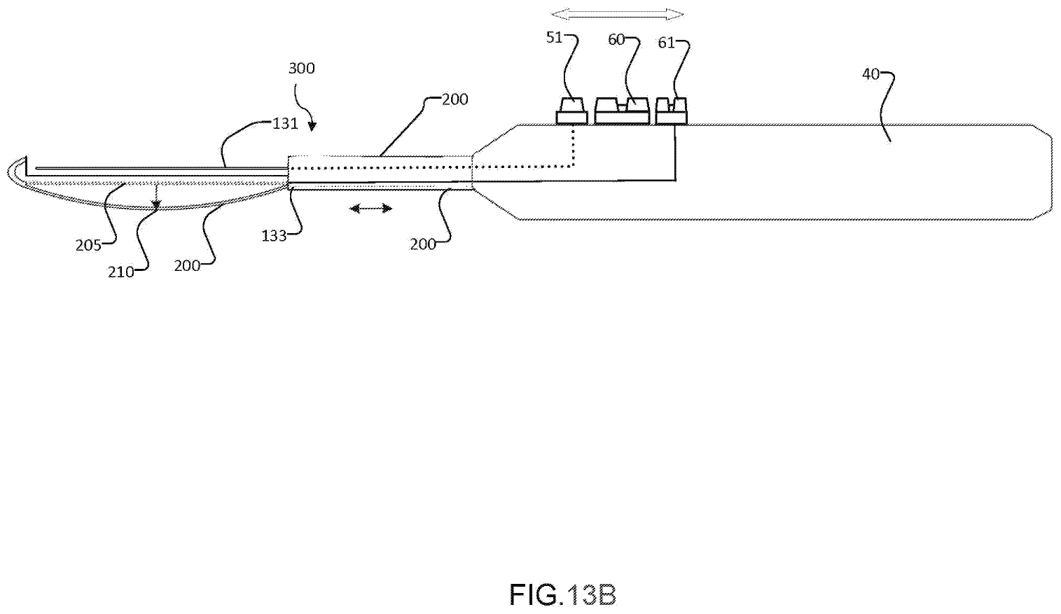

As discussed above, the device 10 may include at least one expansion band 200 that generally surrounds the distal end 30 of the device 10 (see FIGS. 3, 13A, and 13B). Although the features of FIGS. 13 and 13B are described with respect to the soft tissue cutting device 10 having electrosurgical components, similar features may be utilized with the soft tissue cutting device having the blade 130, unless expressly indicated otherwise. In FIG. 13A, a top down view, it is shown that two expansion bands 200 may move laterally from opposing sides of the device through, for example, the shaft openings 120 in side surfaces 90.

FIG. 13B illustrates a side view where a single expansion band 200 is movable between a retracted configuration 205 and an expanded configuration 210. As illustrated, the expanded configuration 210 of expansion band 200 is shown in solid lines and the retracted configuration 205 is shown in dashed lines for ease of understanding. In FIG. 13B it is shown that a bottom expansion band 200 may move downward with respect to the device through, for example, shaft openings 120 in bottom surface 71, for example.

In disclosed embodiments, each expansion band 200 may comprise plural elongated bands. In some embodiments, each expansion band 200 may comprise only a single elongated band. As shown in FIG. 13A, the side or lateral expansion bands 200 expand radially and laterally outwardly relative to lateral sides of the pair of electrodes 131 when the expansion band 200 is in the expanded configuration 210. In more detail, each lateral expansion band 200 bows outwardly when in the expanded configuration 210 so as to expand laterally away from the pair of electrodes 131 and push tissue away from lateral sides of the pair of electrodes 131. Additionally, the lower expansion band 200 bows outwardly in a direction perpendicular to the bowing direction of the lateral expansion bands when in the expanded configuration 210 so as to expand away from the pair of electrodes 131 in the downward direction and push tissue away from lateral sides of the pair of electrodes 131. For example, the two lateral expansion bands 200 of FIG. 13A expand laterally and the lower expansion band 200 of FIG. 13B expands downwardly relative to the pair of electrodes 131, for example. In some embodiments, the expansion band 200 expands only laterally (and not downwardly) relative to the pair of electrodes 131.

At least one advantage of the expansion band 200 may help to anchor the electrosurgical soft tissue cutting device 10 within the body region to provide stability during cutting of the soft tissue. In addition, when the expansion band 200 is in the expanded configuration 210, it may help to push nearby at-risk structures away from the electrosurgical soft tissue cutting device 10 during operation of electrodes 131. In this way, expansion band 200 may expand the “safe zone” in advance of and while the pair of electrodes 131 performs an intended transection or operation so as to ensure that only the desired soft tissue is cut and that nearby at-risk structures are not cut. In some embodiments, the downward direction expansion of the lower expansion band 200 may move the device 10 and electrodes 131 upward in an opposite direction by pushing against patient tissue and thereby facilitating moving the electrodes 131 into a position to transect a target tissue.

As discussed above, the electrosurgical tissue cutting device 10 may also include at least one actuator. For example, a first actuator 60, also referred to herein as a first bowing actuator, is coupled to the two lateral expansion bands 200 such that the movement of the actuator 60 is configured to move the two lateral expansion bands 200 between the retracted configuration 205 and the expanded configuration 210 as shown in FIG. 13A. In some embodiments, first actuator 60 may also control expansion of the lower expansion band 200. Additionally, electrosurgical tissue cutting device 10 may also include a second actuator 61, configured to move the lower expansion band 200 between the retracted configuration 205 and the expanded configuration 210, as shown in FIG. 13B. In some embodiments, each expansion band 200 may have its own actuator (not illustrated) and a user may selectively expand expansion bands 200 to move electrodes 131 into a desired position and/or move adjacent at risk tissues before cutting and/or to provide structural support for device 10 during a transection.

The embodiment of FIGS. 13A and 13B includes two separately controllable actuators 60, 61. For example, a physician may first expand the safe zone by moving first bowing actuator 60 and causing the two lateral expansion bands 200 to move target tissue laterally away from electrode 131. Next, a physician may move the electrode 131 firmly against the target tissue by moving second bowing actuator 61 and causing the lower expansion band 200 to push downward on patient tissue thereby moving the electrodes 131 firmly against the target tissue. Thereafter, a physician may electrically charge electrodes 131 as explained above.

Similar to the discussion of the actuator 60 above with respect to FIG. 3, the actuators 60, 61 are slidable actuators configured to slide along a portion of the handle 40. A physician can slide the actuators 60 and/or 61 using one or more fingers or thumbs, similar to actuator 51. The actuators 60 and 61 may be configured to slide toward the expansion band 200 such that the actuator 60 contacts an adjacent portion of the expansion band 200 and applies a force to the expansion band 200. In some embodiments, expansion band 200 is operably coupled to actuator 60. When sufficient force is applied to the expansion band 200, the expansion band 200 bows outwardly so as to move the expansion band 200 from the retracted configuration 205 to the expanded configuration 210. In some embodiments, the actuator 60 includes a lock to lock the expansion band 200 in a desired position.

In some embodiments, a physician can deploy the pair of electrodes 131 to the activated position only after the lateral expansion bands 200 have been moved to the expanded configuration 210 by way of actuator 60, for example. This is a safety feature designed to prevent the pair of electrodes 131 from being activated and/or exposed if the expansion band 200 has not been expanded. For example, actuator 60 may be positioned on handle 40 farther towards the distal end 30 than actuator 50. In this way, actuator 60 physically blocks actuator 50 from moving forward and deploying electrodes 131 to the activated position. Additionally, as explained above, electrodes 131 may be prevented from receiving an electrical charge from an ESU when they are not positioned in the activated position. Accordingly, in various embodiments, the electrodes 131 also will not become electrically charged unless actuator 60 and/or 61 has expanded the expansion band 200 and created a safe zone, for example.

As discussed above, the expansion band 200 is not inflatable, and thus does not expand via an inflatable mechanism. Additionally, in various embodiments it is contemplated that expansion band 200 will be substantially non-conductive, i.e., formed of an insulative material.

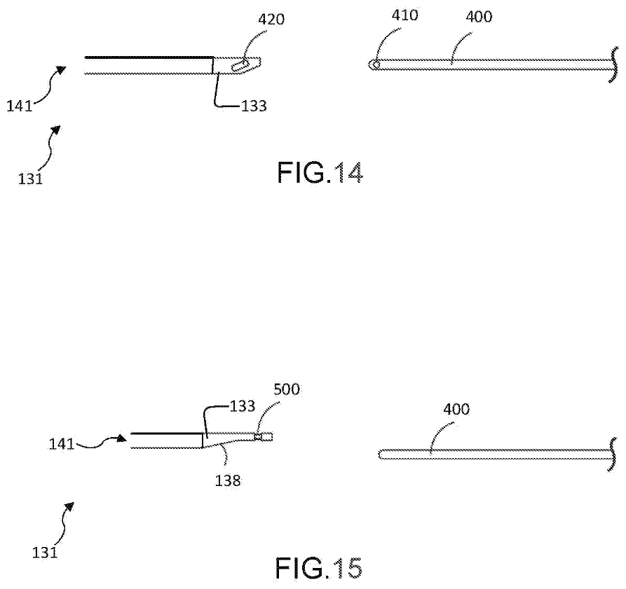

Referring to FIGS. 14 and 15 and similar to as discussed above with respect to FIGS. 6 and 7, the tissue cutting device 10 may also include a push rod 400. The push rod 400 may be positioned in the shaft 70 adjacent the actuator 51, for example. The push rod 400 may comprise metal, plastic, or any other suitable material. In some embodiments, the push rod 400 includes a pin 410, and the body portion 133 includes a slot 420 located in the body portion 133. This arrangement enables the pair of electrodes 131 and body portion 133 to pivot upwardly when the actuator 51 is moved distally, which in turn causes the pair of electrodes 131 to move into the active position as explained hereinabove. This non-limiting embodiment is shown in FIG. 14. However, this feature may be optional, and device 10 may not include this upward pivoting function of electrodes 131.

As shown in FIG. 15, the body portion 133 may include a sloped region 138 that slopes upwardly in a direction toward the projection 500. When the actuator 50 is moved in the distal direction relative to the device 10, the push rod 400 is also pushed distally so as to contact the body portion 133. This in turn causes the push rod 400 to contact and apply a force along the sloped region 138 of the body portion 133. When sufficient force is applied to the body portion 133, a distal end of the pair of electrodes 131 moves upward to move the working end 141 of the pair of electrodes 131 into the active position. Thereafter, a physician may electrically charge electrodes 131 when desired.

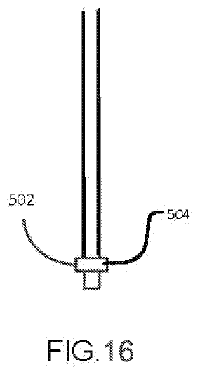

FIG. 16 depicts the electrodes 131 extending from opposing ends 502, 504 of the pivot point projection. In some cases, the electrodes 131 may extend from the ends 502, 504, in a manner similar to the body 135 shown in FIG. 9, discussed above.

As discussed above, suction may be applied to the suction openings to suck fluid, gases, and/or solids suspended in fluid through the suction openings. Furthermore, in some embodiments, tissue may be vaporized due to performing the electrosurgery and the vapor may be evacuated by the suction openings.

FIG. 17 is a schematic view of an electrosurgery system 1100. The electrosurgery system 1100 may comprise an RF Electrosurgical Unit 1102 that is coupled to a power source 1101. Power source 1101 may be any power source and may include any necessary conversion equipment to switch from alternating current to direct current, for example. The RF Electrosurgical Unit 1102 may be electrically coupled to an electrosurgery device 1103. Electrosurgery device may be, for example, devices 10 and/or 700 as disclosed herein. For example, the electrosurgical Unit 1102 may be coupled to devices 10 and/or 700 at connection port 25 (see FIG. 1). In some embodiments, an ultrasound device 1105 may be further provided. Ultrasound devices are well known in the art and can comprise any conventional ultrasound device. For example, GE's Logiq series ultrasound devices, GE Voluson series ultrasound devices, Samsung's Ugeo series ultrasound devices, Alpinion Ecube series ultrasound devices, etc. In practice, a physician such as a surgeon may perform a CTR procedure by inserting a distal end of the electrosurgery device 1103 into an intended surgery site of a patient. Concurrently, a surgeon may guide the distal end of the electrosurgery device 1103 with the usage of ultrasound images obtained from ultrasound device 1105, for example. The physician may expand the “safe-zone” by activating an expansion band 200 or by expanding at least bowing flange 730 consistent with the above disclosure. Once the “safe-zone” has been expanded the surgeon may position a working end 141 of a pair of electrodes 131 in an activated position. At this time, a surgeon may verify that the working end 141 of the pair of electrodes 131 is in the appropriate position by checking diagnostic images obtained by the ultrasound device 1105. Thereafter, a surgeon may electrically charge the pair of electrodes 131 and complete a circuit through the desired tissue of the patient. A surgeon may charge the pair of electrodes 131 by depressing a foot pedal operably coupled to the RF Electrosurgical Unit 1102. In some embodiments, a surgeon may depress a button, actuator, or the like on the electrosurgery device 1103 instead of depressing a foot pedal or the like that is associated primarily with the RF electrosurgical unit 1102. In some embodiments, the working end 141 will transect a tissue as well as cauterize the tissue to minimize bleeding, for example.

In various embodiments, disclosed components of the cutting device 10 (e.g., the cutting device 10 having the blade 130 and/or electrodes 131) may be considered “echogenic” in that they exhibit properties that increase their visibility during ultrasonic imaging, for example. At least some components that may exhibit echogenic properties may include the top surface 80, tip 110, the blade 130, the pair of electrodes 131, a portion of electrodes 131 immediately adjacent working end 141, body portion 133, body shaft 135, and/or expansion band 200, for example. In some embodiments, the top surface 80, tip 110, blade 130, pair of electrodes 131, body portion 133, blade shaft 135, and/or expansion band 200, may be coated with an echogenic coating that increases the visibility of the coated component during ultrasonic imaging, for example.

In at least one embodiment, the top surface 80, blade 130, pair of electrodes 131, and expansion band 200 exhibit echogenic properties. In this embodiment, a surgeon can easily reference an ultrasound in real time and verify a relative distance from the surface 80 to the expansion band 200 in an expanded or semi-expanded position. When using an electrosurgical cutting device 10, this may assist the surgeon in carefully positioning the working end 141 of the pair of electrodes 131 while performing a transection, coagulation, desiccation, or fulguration, for example. In some embodiments, top surface 80 and pair of electrodes 131 may have echogenic line markings spaced evenly at a known interval, e.g., about 2 mm to about 4 mm. These echogenic line markings may provide a surgeon with a “scale” from which to discern a relative size and/or distance of a target tissue to be cut, for example. In the disclosed example, a surgeon may visually identify these evenly spaced line markings as bright portions having a dark portion therebetween. This may assist the surgeon in assessing a relative distance from the cutting edge to a tissue of interest inside of the patient's body, for example. In some embodiments, the blade 130 and/or pair of electrodes 131 may include first multiple echogenic line markings sequentially spaced apart along a longitudinal length thereof at a predetermined interval and the expansion band 200 include second multiple echogenic line markings sequentially spaced apart at the same predetermined interval.

In various embodiments, example disclosed components may exhibit echogenic properties due to being coated in an echogenic coating and/or having an echogenic structure comprising an etching and/or textured surface. An echogenic coating may be resistant to decomposing due to abrasion and may also be biocompatible. In some embodiments, various coatings of different properties may be selectively applied to disclosed components, e.g., a coated portion adjacent a non-coated portion or in alternating sequence of coated and uncoated portions. In some embodiments, such coatings may include microparticles having, for example, a spherical shape. Diameters of various microparticles may range from about 100 nm to about 1000 um, for example. Microparticles may increase the echogenic properties of the component they are disposed on and may also be coated in a lubricating agent such that the microparticles do not inadvertently increase the friction and abrasion to a patient's tissue. An echogenic structure may comprise an etching, a patterned surface, cavities, solid microparticles, hollow bubble like microparticles, substantially flat surfaces, and various combinations and alternating patterns thereof.

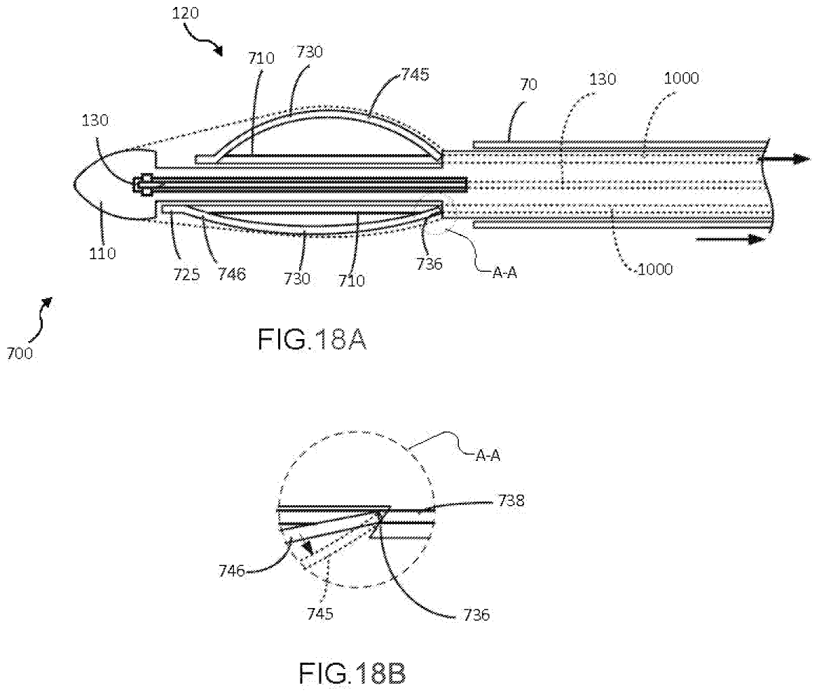

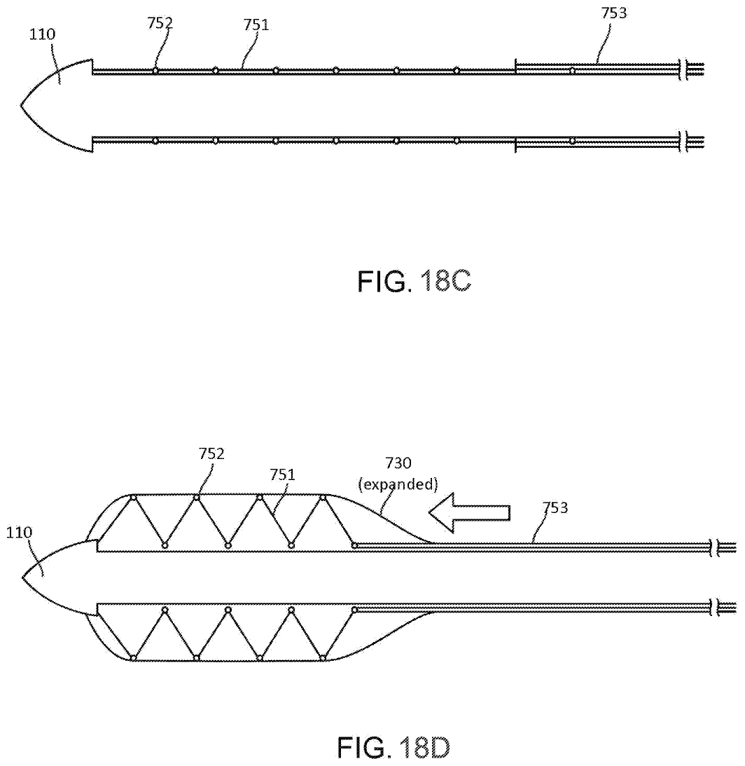

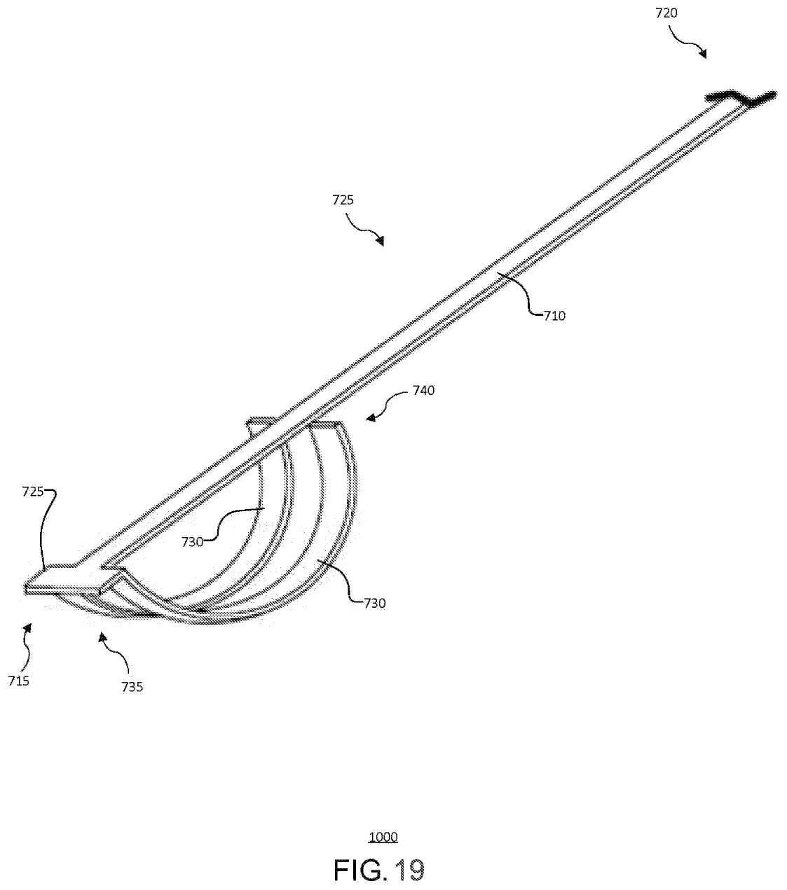

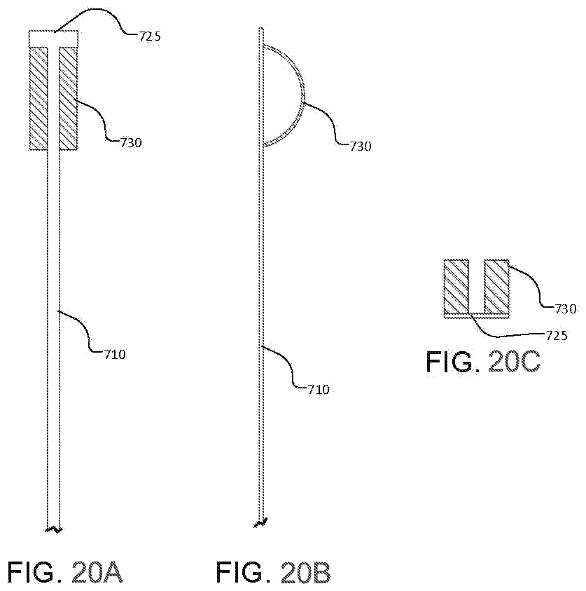

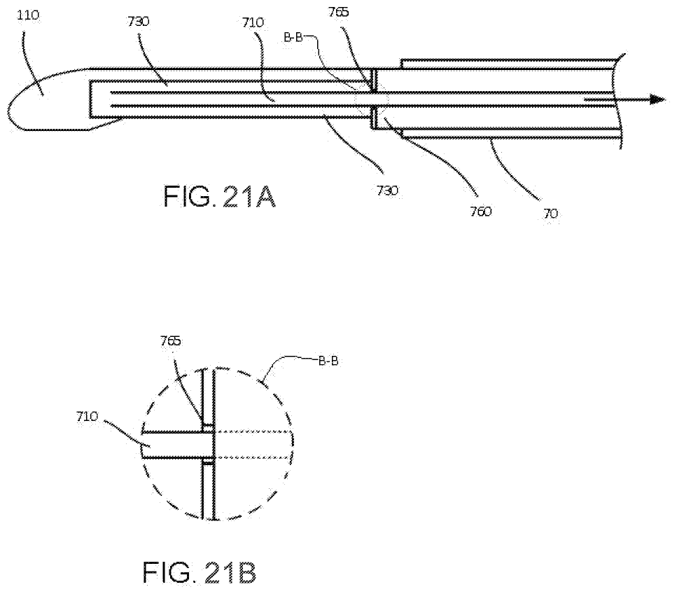

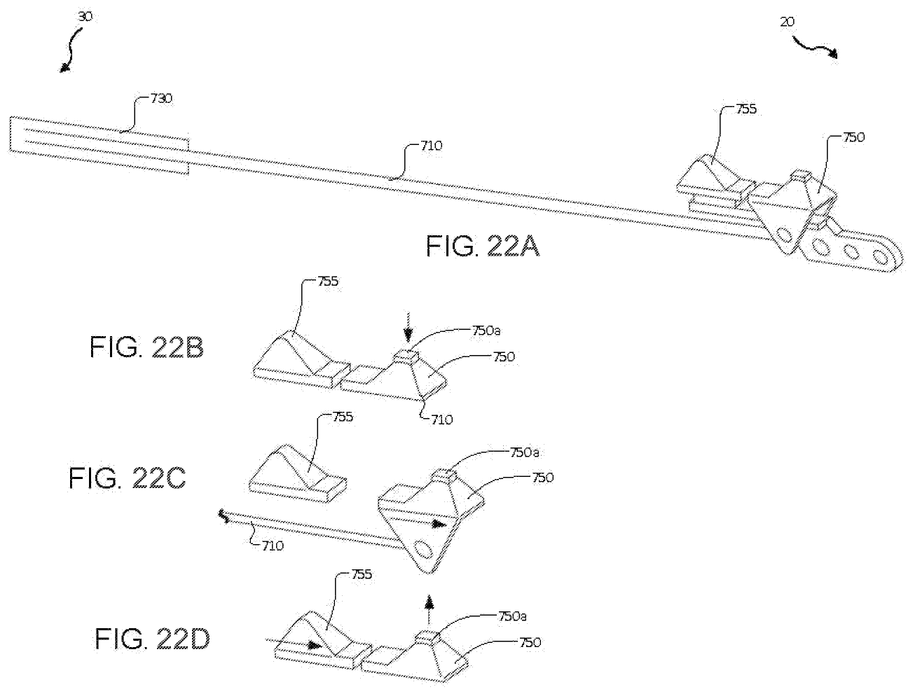

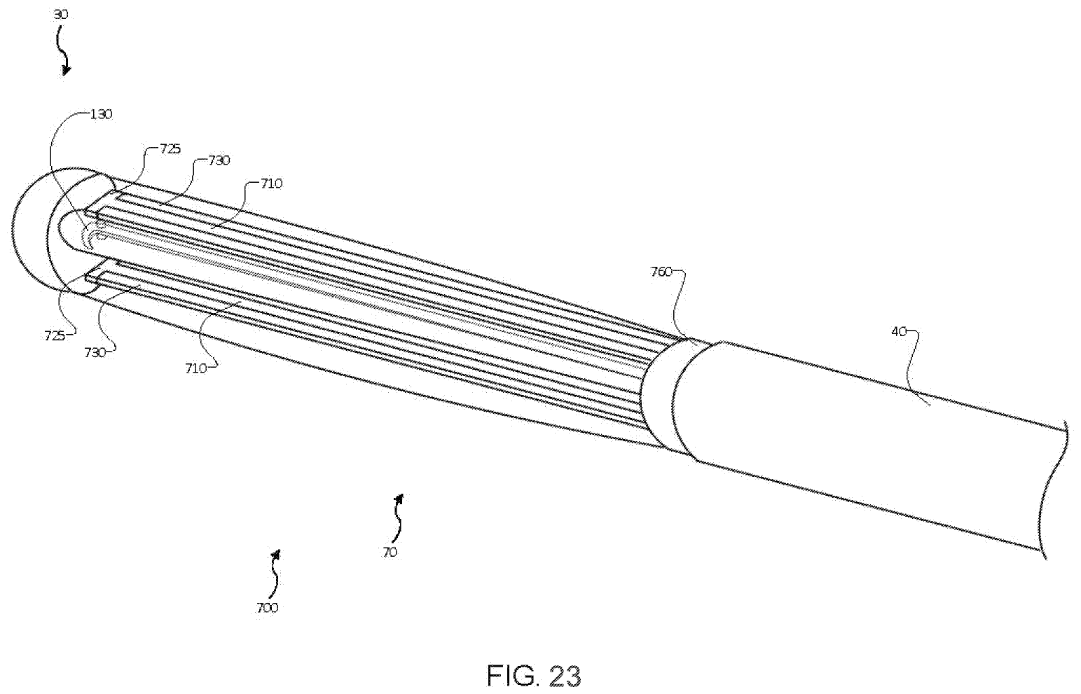

Referring generally to FIGS. 18A-23, a soft tissue cutting device 700 incorporating a multiple bowing flanges 730 is disclosed. Although the soft tissue cutting device 700 is disclosed as having a blade 130, the soft tissue cutting device 700 may, additionally or alternatively, include one or more electrodes (e.g., the electrodes 131, as discussed herein, and/or other suitable electrodes).

Bowing flanges 730 may be used in example embodiments in place of the expansion band 200 disclosed hereinabove. The soft tissue cutting device 700 may have the same, substantially the same, and/or similar components as soft tissue cutting device 10 discussed above. For example, components in each device may be cross compatible unless the context clearly indicates otherwise. Similar to soft tissue cutting device 10, soft tissue cutting device 700 may comprises a shaft 70 that has multiple surfaces and also extends longitudinally along a longitudinal axis. The shaft 70 of soft tissue cutting device 700 includes a shaft opening 120 that extends for a distance along at least one of the multiple surfaces.

Soft tissue cutting device 700 may also include a cutting blade 130 (and/or electrodes 131) that extends through and withdraws from the shaft opening 120 similarly as explained above. The cutting blade 130 of tissue cutting device 700 can have the same features and properties as the cutting blade of tissue cutting device 10. For example, the cutting blade 130 of tissue cutting device 700 may be housed within the shaft 70 when the cutting blade 130 is in an inactive position and the cutting blade 130 may extend through the shaft opening 120 when the cutting blade 130 is in an active position.

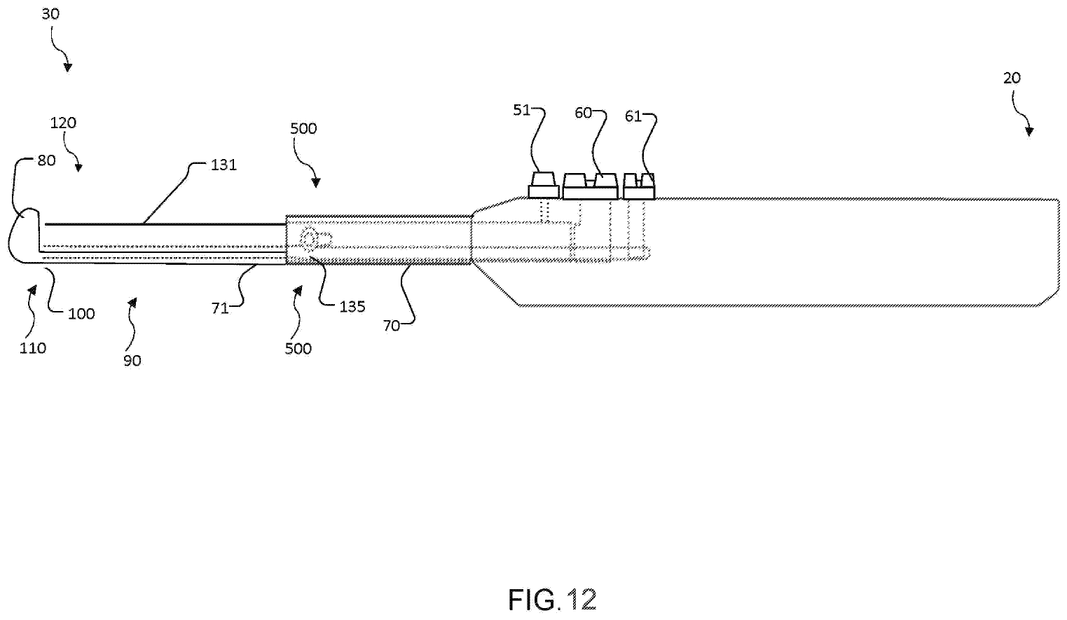

Soft tissue cutting device 700 may further include a bowing system 1000 (see FIG. 12) that includes an elongated support 710 and a pair of bowing flanges 730. The elongated support 710 has a first end 715, a second end 720, and a main portion 725. The main portion 725 extends between the first end 715 and the second end 720. The first end 715 has a pair of tabs 725. Each tab 725 projects laterally away from the main portion 725 of the elongated support 710. The elongated support 710 can comprise a single, unitary structure, such as a single piece of metal or plastic. The elongated support 710 can be formed by any suitable method (e.g., by cutting, stamping, or printing the design). In the illustrated embodiment, the blade 130 is centrally disposed within the shaft 70 relative to the bowing flanges 730. However, in other embodiments (not illustrated) the blade 130 may be alternately disposed. For example, the blade 130 may be disposed on the outside of each pair of bowing flanges 730. For example, still, the blade 130 may be flexible and coupled to the bowing flanges 730 and form a blade working end 140 corresponding in size and position to the bowing flanges 730. In this example, the bowing flanges 730 may expand and contract and so may the flexible blade.

When the cutting device 700 include electrodes 131 in addition to or as an alternative to the blade 130, the pair of electrodes 131 may be disposed on the outside of each pair of bowing flanges 730. For example, the electrodes 131 may comprise a pair of conductive flexible wires that are coupled to the bowing flanges 730 and form a working end 141 corresponding in size and position to the bowing flanges 730. In the example, the bowing flanges may expand and contract and so may the flexible electrodes 131. At least one advantage of this arrangement is that the electrodes may be pushed against surrounding tissue to be transected and/or used to fuse or coagulate the surrounding tissue after a transection has been performed by a pair of centrally disposed electrodes 131 (e.g., electrodes 131, depicted and discussed herein).

The bowing flanges 730 each have a distal end 735 and a proximal end 740. Each bowing flange 730 is movable between an expanded configuration 745 and a retracted configuration 746. The bowing flanges 730 are configured to bow outwardly relative to lateral sides of the cutting blade 130 when the bowing flanges 730 are in the expanded configuration 745. For example, the bowing flanges expand outwardly from the side of cutting device 700.

In disclosed embodiments, the bowing flanges 730 do not expand via an inflatable mechanism. Rather, in such embodiments, the bowing flanges 730 are made from a material that is deformable so as to move the bowing flanges 730 into the expanded configuration 745 when a sufficient contact force, e.g., compression, is applied thereto. When the force ceases to be applied to the bowing flanges 730, the bowing flanges 730 return to the retracted configuration 746 (a neutral resting position). In some embodiments, the bowing flanges 730 are made from a flexible and moldable metal or plastic. However, it is envisioned that alternative materials can also be used for the bowing flanges 730, e.g., an elastomeric material or webbing.

As shown in FIG. 11A and 11B, a compressive force may be applied to flanges 730 to cause lateral expansion of flanges 730. As illustrated, the distal end 735 of each bowing flange 730 may be attached to a respective tab 725 of the elongated support. The proximal end 740 of each bowing flange 730 may be configured to rest on a base 736 of a distal tip portion 738 of the shaft 70 such that contact between the bowing flanges 730 and the base 736 provides stability to the bowing flanges 730 when the bowing flanges 730 are activated (i.e., moved to the expanded configuration 745). Additionally, in some embodiments, base 736 may be a fulcrum point or a pressure point where an internal compressive force is applied to flanges 730. For example, in some embodiments, flanges 730 may be moved from a non-expanded position (where tab 725 is disposed proximate the distal end 30) to an expanded position (where tab 725 and flanges 730 are moved in the proximate direction). By moving flanges 730 in the proximate direction flanges 730 experience an internal compressive force due to contacting base 736. Due to the structural geometry of the bowing system 1000, the internal compressive force may cause each respective flange 730 to bow outwardly (see FIG. 11A). In other embodiments, flanges 730 may experience an internal compressive force due to being moved in a distal direction against a surface similar to base 736. This may cause a similar result and/or expansion as previously explained.

It should be understood that while FIGS. 11A and 11B illustrates the use of a compressive force to move the bowing flanges into an expanded configuration, the disclosure is not so limiting. Other mechanisms may be used for moving the bowing flanges between the retracted configuration and the expanded configuration including, for example, multiple pivotably interconnected plates 751, that can pivot across the hinges 752, and are disposed between the bowing flanges and the base. The interconnected plates 751 can move between a straight configuration (bowing flanges in the retracted configuration) and a hinged configuration (bowing flanges in the expanded configuration), as shown in FIGS. 11C and 11D, respectively. Such interconnected plates may, optionally, be coupled to a mechanism (e.g., actuator 753) for pushing them from the straight configuration to the hinged configuration. Similarly, other mechanisms including, for example, a relatively flat structure having a negligible width and non-negligible dimensions in a direction perpendicular to the width may be provided underneath the bowing flanges. The relatively flat structure may rotate between a first position with the negligible width between the bowing flanges and the base (bowing flanges in the retracted configuration) to a second position with the non-negligible width between the bowing flanges and the base (bowing flanges in the retracted configuration).

In some embodiments, soft tissue cutting device 700 includes a pair of covers (not illustrated). In embodiments of this nature, each cover is located over and surrounds a respective one of the bowing flanges. Optionally, each cover entirely surrounds the respective bowing flange. The covers are configured to cover the bowing flanges to prevent tissue from becoming entrapped in the gap formed between each bowing flange and the elongated support when the bowing flanges 730 are in the expanded configuration. In some embodiments, a single cover may span both bowing flanges 730. The covers can be formed of any medically-compliant material, such a medically-compliant nylon, for example.

Referring to FIGS. 14A and 14B, soft tissue cutting device 700 includes a tip 110. The tip 110 may correspond to a distal-most end of soft tissue cutting device 700. Both lateral sides of the shaft 70 may have a recessed area 760 extending between the tip 110 and the non-recessed area of shaft 70. The recessed area 760 define lateral insertion slots 765 of the soft tissue cutting device 700. Consistent with the disclosure herein, the bowing system 1000 may be configured to be placed into the lateral insertion slots 765, and the bowing flanges 730 may be configured to bow outwardly from the lateral insertion slots 765 when the elongated support 710 is moved in a direction away from the tip 110. As described above, the bowing flanges 730 may be stabilized in the lateral insertion slots 765 when the bowing flanges 730 are in the expanded configuration 745.

Referring to FIGS. 15A-15D, soft tissue cutting device 700 may also include a bowing actuator 750. The bowing actuator 750 may be configured to move the bowing flanges 730 between the expanded configuration 745 and the retracted configuration 746 as previously explained. In some embodiments, the bowing actuator 750 is a slidable actuator attached to the elongated support 710. In such embodiments, the bowing flanges 730 may move to the expanded configuration 745 when the bowing actuator 750 is moved in a proximal direction. By applying tension to the support 710 in a proximal direction (i.e., by pulling the support 710 in a direction away from the tip 110), the tension force is transferred through the tabs 725 as an internal compressive force causing the bowing flanges 730 to bow outwardly relative to the cutting blade 130. The bowing flanges 730 may be deactivated to the non-expanded position by moving the bowing actuator 750 toward the distal end 30, which moves the support 710 toward the tip 110 as well.

Soft tissue cutting device 700 may also include a blade actuator 755. Blade actuator 755 may be configured to move cutting blade 130 between the active position and the inactive position. In some embodiments, the cutting blade 130 is restricted from being in the active position when the bowing flanges 730 are in the retracted configuration 746. This can be accomplished by providing both the blade actuator 755 and the bowing actuator 750 as slidable actuators arranged in a linear side by side configuration. For example, as shown in FIGS. 15B-15D, bowing actuator 750 must first be moved in the proximal direction 20 in order for blade actuator 755 to subsequently be moved in the proximal direction. In such disclosed embodiments, the bowing actuator 750 can be positioned to “block” the blade actuator 755 (i.e., prevent the blade actuator 755 from sliding into a position where the blade 130 may be activated) until the bowing actuator 750 is moved out of its path. This is a safety feature designed to prevent the cutting blade 130 from being deployed (i.e., exposed) if the bowing flanges 730 have not been expanded first. Either or both of the bowing actuator 750 and the blade actuator 755 can include a lock configured to lock the bowing flanges 730 and the blade 130, respectively, in a desired position. For example, bowing actuator 750 includes a push button lock 750a. Both actuators 750, 755 can optionally be devoid of a lock and/or include a lock.

FIG. 16 illustrates an embodiment utilizing a bowing system 1000 comprising a first pair of bowing flanges 730 and a second pair of bowing flanges 730, opposite the first pair of bowing flanges 730. A cutting blade 130 may be disposed within a sheath or shaft and extend between the first and second pairs of bowing flanges. The cutting blade may be configured to perform a transection in a substantially vertical plane, and the bowing flanges may be configured to bow outwardly in a lateral plane. In the illustrated embodiment, the lateral plane is substantially perpendicular to vertical plane.

Some embodiments provide a soft tissue cutting method. The method can use any of the soft issue cutting devices described herein. In one embodiment, the method includes the step of providing a soft tissue cutting device comprising: (a) a cutting blade; and (b) a single expansion band that bows outwardly relative to lateral sides of the cutting blade or alternatively at least one bowing flange that bows outwardly relative to lateral sides of the cutting blade. The method can further include the steps of advancing the soft tissue cutting device to a body region, expanding the expansion band radially outward, and subsequently pivoting the cutting blade upward to expose a cutting surface of the cutting blade so as to cut soft tissue.

Other embodiments provide a method of cutting a transverse carpal ligament. The method can use any of the cutting devices described herein. In one embodiment, the method includes the step of providing a cutting device having an inactive position and an active position. In the inactive position, the cutting device includes: 1) a blade that is housed within a shaft of the cutting device; and 2) an expansion band (or bowing flange) that is in a retracted configuration. In the active position, the expansion band is in an expanded configuration, and the blade extends through an opening in the shaft of the cutting device. The method can further include the steps of advancing the device to a carpal tunnel region while the device is in the inactive position, and cutting a transverse carpal ligament while the device is in the active position.

In some cases, a method for cutting soft tissue may involve the use of any of the device embodiments and features described above. In one example, the method may involve advancing the shaft of an electrosurgical soft tissue cutting device of this disclosure into a body region, expanding one or more expansion bands of the device radially outward, and pivoting the pair of electrodes upward to expose a cutting surface of the pair of electrodes to cut soft tissue. In another embodiment, the method involves advancing the electrosurgical soft tissue cutting device of this disclosure to a body region, expanding at least one bowing flange radially outward and/or downward, and subsequently pivoting the pair of electrodes upward to expose a cutting surface of the pair of electrodes so as to cut soft tissue. Some embodiments may involve moving electrodes from an active position to an inactive position before charging the electrodes.

In the active position, the expansion band is in an expanded configuration, and the pair of electrodes may extend through an opening in the shaft of the electrosurgery device. The method can further include the steps of advancing the device to a carpal tunnel region while the device is in the inactive position, and cutting a transverse carpal ligament by forming a circuit through a patient's tissue via the pair of electrodes while the device is in the active position.

Each of disclosed soft tissue cutting devices 10, 700 may be used in, but is not limited to, transecting the transverse carpal ligament (TCL) during a carpal tunnel release (CTR) surgery. In such embodiments, the physician inserts device 10, 700 through an incision in the wrist into the carpal tunnel region (safe zone) deep to the TCL. This can be done via direct visualization, endoscopic guidance, ultrasound guidance or other imaging guidance systems. Once the device 10, 700 is confirmed to be in the correct surgical position, a corresponding actuator 60, 750 may be slid distally to move the expansion band 200 or bowing flange system 1000 to the expanded configuration. In some embodiments, the corresponding actuator 60, 750 may be locked into position. Next, the corresponding blade actuator 50, 755 may be slid distally to push the shaft and expose the cutting blade 130 to perform a cut or transection. In some embodiments, the corresponding actuator 50, 755 may be locked into position. Once the cutting blade 130 is in the active position and the tip of the cutting blade 130 is in direct contact with a desired tissue (e.g., TCL), the device 10, 700 can pull, push, or pivot the blade 130 to transect the TCL in a distal to proximal direction. When the transection is complete, the device 10, 700 can be deactivated by moving both corresponding actuators 50 and 60 or 755 and 750 in a proximal direction, and removing the device 10, 700 from the body through the wrist incision.

It will be understood that terms such as “same,” “equal,” “planar,” or “coplanar,” as used herein when referring to orientation, layout, location, shapes, sizes, amounts, or other measures do not necessarily mean an exactly identical orientation, layout, location, shape, size, amount, or other measure, but are intended to encompass nearly identical orientation, layout, location, shapes, sizes, amounts, or other measures within acceptable variations that may occur, for example, due to manufacturing processes. The term “substantially” may be used herein to emphasize this meaning, unless the context or other statements clearly indicate otherwise. For example, items described as “substantially the same,” “substantially equal,” or “substantially planar,” may be exactly the same, equal, or planar, or may be the same, equal, or planar within acceptable variations that may occur, for example, due to manufacturing processes and/or tolerances. The term “substantially” may be used to encompass this meaning, especially when such variations do not materially alter functionality.

Various modifications may be made to the embodiments disclosed herein. Likewise, the above disclosed methods may be performed according to an alternate sequence. Therefore, the above description should not be construed as limiting, but merely as exemplification of the various embodiments.

1. A soft tissue cutting device, comprising:
a handle;
a shaft extending from a proximal end coupled with the handle to a distal end;
a shaft opening disposed along a distal portion of a top surface of the shaft;
a blade that extends through the shaft opening and moves proximally toward the handle to cut soft tissue;
an expansion member on the distal portion of the shaft, wherein the expansion member is configured to expand in at least two directions from the shaft; and
a first actuator on the handle for moving the blade proximally to cut the soft tissue.

2. The soft tissue cutting device of claim 1, wherein the first actuator moves the blade from an inactive position within the shaft to an active position in which the blade is exposed through the shaft opening.

3. The soft tissue cutting device of claim 2, wherein the first actuator comprises a first slider on a top surface of the handle.

4. The soft tissue cutting device of claim 3, further comprising a second actuator on the handle for expanding the expandable member.

5. The soft tissue cutting device of claim 4, wherein the second actuator comprises a second slider.

6. The soft tissue cutting device of claim 2, further comprising a push rod in the shaft, configured to move the blade between the active position and the inactive position.

7. The soft tissue cutting device of claim 6, further comprising:
a blade shaft extending to the blade and comprising a slot; and
a pin extending through the push rod and into the slot, wherein the blade is configured to pivot when the pin rotates within the slot.

8. The soft tissue cutting device of claim 1, wherein the expansion member comprises an expansion band that bows outwardly from the distal portion of the shaft.

9. The soft tissue cutting device of claim 8, further comprising a second actuator on the handle to expand and contract the expansion band.

10. (canceled)

11. The soft tissue cutting device of claim 1, wherein the blade is movable independently of the expansion member.

12. A method of cutting a transverse carpal ligament in a hand of a patient, the method comprising:
advancing a distal portion of a cutting device to a carpal tunnel region while the cutting device is in an inactive position;
expanding an expansion member on the distal portion of the cutting device to cause it to expand and thus form a safe zone;
adjusting a first actuator on a handle of the cutting device to perform the following steps:
moving a blade on the distal portion of the cutting device from an inactive position to an active position;
cutting the transverse carpal ligament with the blade; and
moving the blade back to the inactive position; and
removing the cutting device from the hand.

13. An electrosurgical soft tissue cutting device, comprising:
a handle;
a shaft extending from the handle, the shaft comprising:
a proximal portion,
a distal portion,
an upper surface,
a lower surface, and
a first shaft opening that extends longitudinally along the upper surface of at least part of the distal portion of the shaft;
a pair of electrodes configured for cutting soft tissue; and
a first expansion band proximate the distal portion of the shaft, the first expansion band being movable between an expanded configuration in which the first expansion band bows outwardly in a lateral direction, and a retracted configuration.

14. The electrosurgical soft tissue cutting device of claim 13, wherein in the expanded configuration the first expansion band bows outwardly relative to lateral sides of a working end of the pair of electrodes.

15. The electrosurgical soft tissue cutting device of claim 13, further comprising:
a second expansion band proximate the distal portion of the shaft that bows outwardly in an opposite direction relative to the first expansion band, the first and second expansion bands being movable between respective expanded configuration and the retracted configurations,
wherein, in the expanded configuration, the first expansion band and the second expansion band bow outwardly relative to lateral sides of the working end of the pair of electrodes in opposing directions.

16. The electrosurgical soft tissue cutting device of claim 15, further comprising an electrode actuator on the handle configured to selectively electrically charge the pair of electrodes between an electrically charged state and non-charged state, wherein the electrode actuator is a slidable actuator.

17. The electrosurgical soft tissue cutting device of claim 16, further comprising a first expansion band actuator selectively moveable between an activated position and a non-activated position, wherein the activated position causes the first expansion band and the second expansion band to move into the expanded configuration.

18. The electrosurgical soft tissue cutting device of claim 17, wherein the electrode actuator may only charge the pair of electrodes when the first expansion band actuator is in the activated position.

19. The electrosurgical soft tissue cutting device of claim 18, comprising:
a second shaft opening that extends longitudinally along the lower surface of at least part of the distal portion of the shaft; and
a third expansion band proximate the distal portion of the shaft, beneath the first expansion band and second expansion band, the third expansion band being movable between a lower expanded configuration and a lower retracted configuration,
wherein, in the lower expanded configuration, the third expansion band bows outwardly and downward relative to the working end of the pair of electrodes through the second shaft opening.

20. The electrosurgical soft tissue cutting device of claim 19, further comprising a second expansion band actuator configured to move the third expansion band between the lower expanded configuration and the lower retracted configuration.

21. The electrosurgical soft tissue cutting device of claim 20, wherein the electrode actuator is configured to charge the pair of electrodes when the first expansion band actuator and second expansion band actuators are in the activated position.

22. The electrosurgical soft tissue cutting device of claim 13, wherein each of the electrodes of the pair of electrodes is joined to the other a rigid insulating member therebetween.

23. The electrosurgical soft tissue cutting device of claim 22, wherein the pair of electrodes is configured for bipolar radio frequency soft tissue cutting methods.

24. A method of cutting a transverse carpal ligament in a patient, the method comprising:
advancing a shaft of an electrosurgical device into a hand of the patient to position a distal end of the device at or near the transverse carpal ligament, the electrosurgical device comprising:
a pair of electrodes at or near the distal end of the shaft, and
a pair of lateral expansion bands on the shaft;
expanding the pair of lateral expansion bands such that each expansion band of the pair of expansion bands bows radially outwardly relative to lateral sides of the pair of electrodes in opposite directions;
positioning the shaft such that the transverse carpal ligament lies within or adjacent to the pair of electrodes; and
electrically charging the pair of electrodes to transect the transverse carpal ligament.

25. The method of claim 24, further comprising expanding a lower expansion band of the electrosurgical device to cause it to bow outwardly from the shaft in a downward direction and push the pair of electrodes against the transverse carpal ligament.

26. The method of claim 25, wherein expanding the pair of lateral expansion bands and the lower expansion band comprises actuating at least one expansion band actuator on a handle of the electrosurgical device.

27. The method of claim 24, wherein electrically charging the pair of electrodes comprises actuating an electrode actuator on a handle of the electrosurgical device.

28. The method of claim 27, wherein each of the pair of electrodes is a bipolar radiofrequency electrode, and wherein electrically charging the pair of electrodes comprises sending a radiofrequency signal from one of the pair of electrodes, through the transverse carpal ligament, to the other of the pair or electrodes.
